SEQUENCE LISTING (179 sequences)

SEQ ID NO: 1 - DNA, 22 bp, Homo sapiens
ctatacagtctactgtctttcc

SEQ ID NO: 2 - DNA, 23 bp, Homo sapiens
aaaagtgcttacagtgcaggtag

SEQ ID NO: 3 - DNA, 20 bp, Homo sapiens
tcactgttcagacaggcgga

SEQ ID NO: 4 - DNA, 20 bp, Homo sapiens
tgagccctgtcctcccgcag

SEQ ID NO: 5 - DNA, 22 bp, Homo sapiens
aactggatcaattataggagtg

SEQ ID NO: 6 - DNA, 22 bp, Homo sapiens
atgggtgaatttgtagaaggat

SEQ ID NO: 7 - DNA, 21 bp, Homo sapiens
cctgttgaagtgtaatcccca

SEQ ID NO: 8 - DNA, 20 bp, Homo sapiens
taaagagccctgtggagaca

SEQ ID NO: 9 - DNA, 22 bp, Homo sapiens
tctgggcaacaaagtgagacct

SEQ ID NO: 10 - DNA, 19 bp, Homo sapiens
tggatttttggatcaggga

SEQ ID NO: 11 - DNA, 22 bp, Homo sapiens
ttcattcggctgtccagatgta

SEQ ID NO: 12 - DNA, 22 bp, Homo sapiens
ttttcaactctaatgggagaga

SEQ ID NO: 13 - DNA, 21 bp, Homo sapiens
taccacagggtagaaccacgg

SEQ ID NO: 14 - DNA, 23 bp, Homo sapiens
tgtagtgtttcctactttatgga

SEQ ID NO: 15 - DNA, 22 bp, Homo sapiens
ggatatcatcatatactgtaag

SEQ ID NO: 16 - DNA, 22 bp, Homo sapiens
tgagaactgaattccatgggtt

SEQ ID NO: 17 - DNA, 22 bp, Homo sapiens
tctcccaacccttgtaccagtg

SEQ ID NO: 18 - DNA, 23 bp, Homo sapiens
caaagtgcttacagtgcaggtag

SEQ ID NO: 19 - DNA, 22 bp, Homo sapiens
caaagaattctccttttgggct

SEQ ID NO: 20 - DNA, 23 bp, Homo sapiens
taaagtgcttatagtgcaggtag

SEQ ID NO: 21 - DNA, 22 bp, Homo sapiens
ctgtgcgtgtgacagcggctga

SEQ ID NO: 22 - DNA, 21 bp, Homo sapiens
agctacatctggctactgggt

SEQ ID NO: 23 - DNA, 22 bp, Homo sapiens
tgtcagtttgtcaaatacccca

SEQ ID NO: 24 - DNA, 22 bp, Homo sapiens
tggctcagttcagcaggaacag

SEQ ID NO: 25 - DNA, 22 bp, Homo sapiens
tgcctactgagctgaaacacag

SEQ ID NO: 26 - DNA, 21 bp, Homo sapiens
ttcaagtaattcaggataggt

SEQ ID NO: 27 - DNA, 22 bp, Homo sapiens
ctttcagtcggatgtttgcagc

SEQ ID NO: 28 - DNA, 22 bp, Homo sapiens
tgtaaacatcctcgactggaag

SEQ ID NO: 29 - DNA, 23 bp, Homo sapiens
tgtaaacatcctacactctcagc

SEQ ID NO: 30 - DNA, 22 bp, Homo sapiens
aaaagctgggttgagagggcga

SEQ ID NO: 31 - DNA, 21 bp, Homo sapiens
cacattacacggtcgacctct

SEQ ID NO: 32 - DNA, 22 bp, Homo sapiens
aacaatatcctggtgctgagtg

SEQ ID NO: 33 - DNA, 21 bp, Homo sapiens
gtgcattgtagttgcattgca

SEQ ID NO: 34 - DNA, 20 bp, Homo sapiens
gtgcattgctgttgcattgc

SEQ ID NO: 35 - DNA, 22 bp, Homo sapiens
ttataaagcaatgagactgatt

SEQ ID NO: 36 - DNA, 23 bp, Homo sapiens
tctcacacagaaatcgcacccgt

SEQ ID NO: 37 - DNA, 21 bp, Homo sapiens
actggacttggagtcagaagg

SEQ ID NO: 38 - DNA, 22 bp, Homo sapiens
cagcagcaattcatgttttgaa

SEQ ID NO: 39 - DNA, 22 bp, Homo sapiens
aagacgggaggaaagaagggag

SEQ ID NO: 40 - DNA, 22 bp, Homo sapiens
tcaggctcagtcccctcccgat

SEQ ID NO: 41 - DNA, 22 bp, Homo sapiens
gtcatacacggctctcctctct

SEQ ID NO: 42 - DNA, 22 bp, Homo sapiens
tcctgtactgagctgccccgag

SEQ ID NO: 43 - DNA, 21 bp, Homo sapiens
cggggcagctcagtacaggat

SEQ ID NO: 44 - DNA, 21 bp, Homo sapiens
atccttgctatctgggtgcta

SEQ ID NO: 45 - DNA, 23 bp, Homo sapiens
agtgcctgagggagtaagagccc

SEQ ID NO: 46 - DNA, 23 bp, Homo sapiens
gtttgcacgggtgggccttgtct

SEQ ID NO: 47 - DNA, 20 bp, Homo sapiens
gtccgctcggcggtggccca

SEQ ID NO: 48 - DNA, 19 bp, Homo sapiens
gagccagttggacaggagc

SEQ ID NO: 49 - DNA, 21 bp, Homo sapiens
tcttgtgttctctagatcagt

SEQ ID NO: 50 - DNA, 22 bp, Homo sapiens
taactggttgaacaactgaacc

SEQ ID NO: 51 - DNA, 22 bp, Homo sapiens
ttatggtttgcctgggactgag

SEQ ID NO: 52 - DNA, 22 bp, Homo sapiens
tatgcattgtatttttaggtcc

SEQ ID NO: 53 - DNA, 21 bp, Homo sapiens
aagcctgcccggctcctcggg

SEQ ID NO: 54 - DNA, 22 bp, Homo sapiens
tgtgtcactcgatgaccactgt

SEQ ID NO: 55 - DNA, 22 bp, Homo sapiens
gactatagaactttccccctca

SEQ ID NO: 56 - DNA, 22 bp, Homo sapiens
agtattctgtaccagggaaggt

SEQ ID NO: 57 - DNA, 25 bp, Homo sapiens
agggatcgcgggcgggtggcggcct

SEQ ID NO: 58 - DNA, 19 bp, Homo sapiens
tctaggctggtactgctga

SEQ ID NO: 59 - DNA, 19 bp, Homo sapiens
aagtgtgcagggcactggt

SEQ ID NO: 60 - DNA, 21 bp, Homo sapiens
aatattatacagtcaacctct

SEQ ID NO: 61 - DNA, 23 bp, Homo sapiens
ggcaggttctcaccctctctagg

SEQ ID NO: 62 - DNA, 22 bp, Homo sapiens
cttggttcagggagggtcccca

SEQ ID NO: 63 - DNA, 24 bp, Homo sapiens
tgcctgggtctctggcctgcgcgt

SEQ ID NO: 64 - DNA, 17 bp, Homo sapiens
tctcgctggggcctcca

SEQ ID NO: 65 - DNA, 23 bp, Homo sapiens
tccagtaccacgtgtcagggcca

SEQ ID NO: 66 - DNA, 22 bp, Homo sapiens
tatacctcagttttatcaggtg

SEQ ID NO: 67 - DNA, 22 bp, Homo sapiens
cactggctcctttctgggtaga

SEQ ID NO: 68 - DNA, 22 bp, Homo sapiens
caagctcgtgtctgtgggtccg

SEQ ID NO: 69 - DNA, 22 bp, Homo sapiens
aaaccgttaccattactgagtt

SEQ ID NO: 70 - DNA, 23 bp, Homo sapiens
caaagtgctgttcgtgcaggtag

SEQ ID NO: 71 - DNA, 22 bp, Homo sapiens
tgaggtagtaggttgtatagtt

SEQ ID NO: 72 - DNA, 22 bp, Homo sapiens
agaggtagtaggttgcatagtt

SEQ ID NO: 73 - DNA, 22 bp, Homo sapiens
tgaggtaggaggttgtatagtt

SEQ ID NO: 74 - DNA, 22 bp, Homo sapiens
tgaggtagtagattgtatagtt

SEQ ID NO: 75 - DNA, 22 bp, Homo sapiens
tgaggtagtagtttgtacagtt

SEQ ID NO: 76 - DNA, 23 bp, Homo sapiens
agcagcattgtacagggctatga

SEQ ID NO: 77 - DNA, 23 bp, Homo sapiens
aaaagtgcttacagtgcaggtag

SEQ ID NO: 78 - DNA, 21 bp, Homo sapiens
taaagtgctgacagtgcagat

SEQ ID NO: 79 - DNA, 20 bp, Homo sapiens
taaggcacgcggtgaatgcc

SEQ ID NO: 80 - DNA, 26 bp, Homo sapiens
aagtagttggtttgtatgagatggtt

SEQ ID NO: 81 - DNA, 22 bp, Homo sapiens
tcgtaccgtgagtaataatgcg

SEQ ID NO: 82 - DNA, 21 bp, Homo sapiens
cattattacttttggtacgcg

SEQ ID NO: 83 - DNA, 18 bp, Homo sapiens
atcccacctctgccacca

SEQ ID NO: 84 - DNA, 18 bp, Homo sapiens
gtccctgttcaggcgcca

SEQ ID NO: 85 - DNA, 20 bp, Homo sapiens
taaagagccctgtggagaca

SEQ ID NO: 86 - DNA, 22 bp, Homo sapiens
cagtgcaatgttaaaagggcat

SEQ ID NO: 87 - DNA, 22 bp, Homo sapiens
cagtgcaatgatgaaagggcat

SEQ ID NO: 88 - DNA, 22 bp, Homo sapiens
tttggtccccttcaaccagctg

SEQ ID NO: 89 - DNA, 22 bp, Homo sapiens
tgtgactggttgaccagagggg

SEQ ID NO: 90 - DNA, 22 bp, Homo sapiens
tctacagtgcacgtgtctccag

SEQ ID NO: 91 - DNA, 22 bp, Homo sapiens
cagtggttttaccctatggtag

SEQ ID NO: 92 - DNA, 23 bp, Homo sapiens
tgtagtgtttcctactttatgga

SEQ ID NO: 93 - DNA, 21 bp, Homo sapiens
cataaagtagaaagcactact

SEQ ID NO: 94 - DNA, 22 bp, Homo sapiens
tgagaactgaattccatgggtt

SEQ ID NO: 95 - DNA, 22 bp, Homo sapiens
tgagaactgaattccataggct

SEQ ID NO: 96 - DNA, 22 bp, Homo sapiens
tcagtgcactacagaactttgt

SEQ ID NO: 97 - DNA, 22 bp, Homo sapiens
tcagtgcatcacagaactttgt

SEQ ID NO: 98 - DNA, 21 bp, Homo sapiens
agggagggacgggggctgtgc

SEQ ID NO: 99 - DNA, 22 bp, Homo sapiens
tctcccaacccttgtaccagtg

SEQ ID NO: 100 - DNA, 21 bp, Homo sapiens
tcgaggagctcacagtctagt

SEQ ID NO: 101 - DNA, 22 bp, Homo sapiens
tagcagcacatcatggtttaca

SEQ ID NO: 102 - DNA, 22 bp, Homo sapiens
tagcagcacgtaaatattggcg

SEQ ID NO: 103 - DNA, 23 bp, Homo sapiens
caaagtgcttacagtgcaggtag

SEQ ID NO: 104 - DNA, 18 bp, Homo sapiens
tccagtgccctcctctcc

SEQ ID NO: 105 - DNA, 22 bp, Homo sapiens
tggagagaaaggcagttcctga

SEQ ID NO: 106 - DNA, 22 bp, Homo sapiens
caaagaattctccttttgggct

SEQ ID NO: 107 - DNA, 23 bp, Homo sapiens
taaggtgcatctagtgcagttag

SEQ ID NO: 108 - DNA, 23 bp, Homo sapiens
caacggaatcccaaaagcagctg

SEQ ID NO: 109 - DNA, 21 bp, Homo sapiens
ctgacctatgaattgacagcc

SEQ ID NO: 110 - DNA, 21 bp, Homo sapiens
tagcagcacagaaatattggc

SEQ ID NO: 111 - DNA, 22 bp, Homo sapiens
taggtagtttcctgttgttggg

SEQ ID NO: 112 - DNA, 22 bp, Homo sapiens
acagtagtctgcacattggtta

SEQ ID NO: 113 - DNA, 23 bp, Homo sapiens
tgtgcaaatctatgcaaaactga

SEQ ID NO: 114 - DNA, 23 bp, Homo sapiens
tgtgcaaatccatgcaaaactga

SEQ ID NO: 115 - DNA, 22 bp, Homo sapiens
taatactgcctggtaatgatga

SEQ ID NO: 116 - DNA, 23 bp, Homo sapiens
taaagtgcttatagtgcaggtag

SEQ ID NO: 117 - DNA, 23 bp, Homo sapiens
caaagtgctcatagtgcaggtag

SEQ ID NO: 118 - DNA, 22 bp, Homo sapiens
tagcttatcagactgatgttga

SEQ ID NO: 119 - DNA, 23 bp, Homo sapiens
agctacattgtctgctgggtttc

SEQ ID NO: 120 - DNA, 22 bp, Homo sapiens
tgtcagtttgtcaaatacccca

SEQ ID NO: 121 - DNA, 22 bp, Homo sapiens
cgtgtatttgacaagctgagtt

SEQ ID NO: 122 - DNA, 22 bp, Homo sapiens
tgcctactgagctgaaacacag

SEQ ID NO: 123 - DNA, 22 bp, Homo sapiens
cattgcacttgtctcggtctga

SEQ ID NO: 124 - DNA, 22 bp, Homo sapiens
ttcaagtaatccaggataggct

SEQ ID NO: 125 - DNA, 21 bp, Homo sapiens
ttcaagtaattcaggataggt

SEQ ID NO: 126 - DNA, 21 bp, Homo sapiens
ttcacagtggctaagttccgc

SEQ ID NO: 127 - DNA, 22 bp, Homo sapiens
agggcttagctgcttgtgagca

SEQ ID NO: 128 - DNA, 22 bp, Homo sapiens
aaggagctcacagtctattgag

SEQ ID NO: 129 - DNA, 22 bp, Homo sapiens
tatgtgggatggtaaaccgctt

SEQ ID NO: 130 - DNA, 22 bp, Homo sapiens
tagcaccatctgaaatcggtta

SEQ ID NO: 131 - DNA, 22 bp, Homo sapiens
tagcaccatttgaaatcggtta

SEQ ID NO: 132 - DNA, 23 bp, Homo sapiens
cagtgcaatagtattgtcaaagc

SEQ ID NO: 133 - DNA, 23 bp, Homo sapiens
cagtgcaatgatattgtcaaagc

SEQ ID NO: 134 - DNA, 22 bp, Homo sapiens
tgtaaacatcctacactcagct

SEQ ID NO: 135 - DNA, 23 bp, Homo sapiens
tgtaaacatcctacactctcagc

SEQ ID NO: 136 - DNA, 21 bp, Homo sapiens
aggcaagatgctggcatagct

SEQ ID NO: 137 - DNA, 20 bp, Homo sapiens
actgccccaggtgctgctgg

SEQ ID NO: 138 - DNA, 22 bp, Homo sapiens
ctggccctctctgcccttccgt

SEQ ID NO: 139 - DNA, 23 bp, Homo sapiens
tcaagagcaataacgaaaaatgt

SEQ ID NO: 140 - DNA, 22 bp, Homo sapiens
ttataaagcaatgagactgatt

SEQ ID NO: 141 - DNA, 22 bp, Homo sapiens
tccgtctcagttactttatagc

SEQ ID NO: 142 - DNA, 23 bp, Homo sapiens
tctcacacagaaatcgcacccgt

SEQ ID NO: 143 - DNA, 22 bp, Homo sapiens
taatgcccctaaaaatccttat

SEQ ID NO: 144 - DNA, 22 bp, Homo sapiens
ttataatacaacctgataagtg

SEQ ID NO: 145 - DNA, 22 bp, Homo sapiens
atataatacaacctgctaagtg

SEQ ID NO: 146 - DNA, 21 bp, Homo sapiens
aacatagaggaaattccacgt

SEQ ID NO: 147 - DNA, 22 bp, Homo sapiens
tggttgaccatagaacatgcgc

SEQ ID NO: 148 - DNA, 21 bp, Homo sapiens
aatataacacagatggcctgt

SEQ ID NO: 149 - DNA, 22 bp, Homo sapiens
actggacttagggtcagaaggc

SEQ ID NO: 150 - DNA, 23 bp, Homo sapiens
aatgacacgatcactcccgttga

SEQ ID NO: 151 - DNA, 23 bp, Homo sapiens
tcttggagtaggtcattgggtgg

SEQ ID NO: 152 - DNA, 22 bp, Homo sapiens
aaaccgttaccattactgagtt

SEQ ID NO: 153 - DNA, 23 bp, Homo sapiens
tagtgcaatattgcttatagggt

SEQ ID NO: 154 - DNA, 22 bp, Homo sapiens
tcctgtactgagctgccccgag

SEQ ID NO: 155 - DNA, 22 bp, Homo sapiens
tgaaacatacacgggaaacctc

SEQ ID NO: 156 - DNA, 22 bp, Homo sapiens
aaacaaacatggtgcacttctt

SEQ ID NO: 157 - DNA, 18 bp, Homo sapiens
tgcttcctttcagagggt

SEQ ID NO: 158 - DNA, 22 bp, Homo sapiens
aaaatggttccctttagagtgt

SEQ ID NO: 159 - DNA, 22 bp, Homo sapiens
catgccttgagtgtaggaccgt

SEQ ID NO: 160 - DNA, 22 bp, Homo sapiens
cctcccacacccaaggcttgca

SEQ ID NO: 161 - DNA, 22 bp, Homo sapiens
aaacattcgcggtgcacttctt

SEQ ID NO: 162 - DNA, 21 bp, Homo sapiens
tgagttggccatctgagtgag

SEQ ID NO: 163 - DNA, 22 bp, Homo sapiens
cacgctcatgcacacacccaca

SEQ ID NO: 164 - DNA, 21 bp, Homo sapiens
taattttatgtataagctagt

SEQ ID NO: 165 - DNA, 22 bp, Homo sapiens
gagcttattcataaaagtgcag

SEQ ID NO: 166 - DNA, 22 bp, Homo sapiens
atgctgacatatttactagagg

SEQ ID NO: 167 - DNA, 25 bp, Homo sapiens
agggatcgcgggcgggtggcggcct

SEQ ID NO: 168 - DNA, 22 bp, Homo sapiens
gtccctctccaaatgtgtcttg

SEQ ID NO: 169 - DNA, 21 bp, Homo sapiens
aggaggcagcgctctcaggac

SEQ ID NO: 170 - DNA, 22 bp, Homo sapiens
tttaggataagcttgacttttg

SEQ ID NO: 171 - DNA, 21 bp, Homo sapiens
aatggcgccactagggttgtg

SEQ ID NO: 172 - DNA, 22 bp, Homo sapiens
tacccattgcatatcggagttg

SEQ ID NO: 173 - DNA, 22 bp, Homo sapiens
caacaaatcacagtctgccata

SEQ ID NO: 174 - DNA, 22 bp, Homo sapiens
ctgttgccactaacctcaacct

SEQ ID NO: 175 - DNA, 21 bp, Homo sapiens
tggaggagaaggaaggtgatg

SEQ ID NO: 176 - DNA, 22 bp, Homo sapiens
tatacctcagttttatcaggtg

SEQ ID NO: 177 - DNA, 23 bp, Homo sapiens
caaagtgctgttcgtgcaggtag

SEQ ID NO: 178 - DNA, 22 bp, Homo sapiens
tcttctctgttttggccatgtg

SEQ ID NO: 179 - RNA, 25 nt, Homo sapiens
agggaucgcgggcggguggcggccu

(19) United States
(12) Patent Application Publication
(10) Pub. No.: US 2015/0057173 A1
(43) Pub. Date: Feb. 26, 2015

(54) METHODS FOR DETERMINING THE TROPISM AND RECEPTOR USAGE OF A VIRUS, IN PARTICULAR HIV, IN BODY SAMPLES TAKEN FROM THE CIRCULATION

(71) Applicants: Prestizia, Clapiers (FR); centre National de la Recherche Scientifique, Paris (FR); Universite Montpellier 2 Sciences et Techniques, Montpellier Cedex 5 (FR)

(72) Inventors: Dominique Blanchard, Nantes (FR); Sophie Bonnel, Cournonterral (FR); Valerie Courgnaud, Montpellier (FR); Charles-Henri Lecellier, Les Cres (FR); Marjorie Monleau, Murles (FR)

(73) Assignees: Prestizia, Clapiers (FR); Centre NAtional de la Rechercxhe Scientifique, Paris (FR); Universite Montpellier 2 Sciences et Techniques, Montpellier Cedex 5 (FR)

(21) Appl. No.: 14/385,010

(22) PCT Filed: Mar. 15, 2013
(86) PCT No.: PCT/EP2013/055486

§ 371 Date: Sep. 12, 2014

(30) Foreign Application Priority Data
Mar. 19, 2012 (EP) 12305322.5

(51) Int. Cl. C12Q 1/70

(52) U.S. Cl. CPC C12Q 1/701; C12Q 2600/178; C12Q 2600/16
USPC 506/9; 435/5; 506/16

(57) ABSTRACT

The present invention relates to microarrays, sets of primers and methods for determining the co-receptor usage of a virus, in particular whether HIV uses CXCR4 or CCR5 as co-receptor. The methods and compositions of the invention are based on microRNA analysis. The invention can be used to determine virus tropism in any species or organism that expresses microRNAs, particularly in human beings.

The present invention relates to compositions and methods for determining the tropism of a virus. The methods and compositions of the invention are based on microRNA analysis. The invention can be used to determine virus tropism in any species or organism that expresses microRNAs, particularly in human beings.

### BACKGROUND OF THE INVENTION

MicroRNAs are a class of RNAs involved in post-transcriptional regulation of genes. MicroRNAs are typically single-stranded, of about 10-50 nucleotides in length, preferably between about 15-25 nucleotides, and they regulate the transcription of target genes by degrading or blocking translation of the mRNA. MicroRNAs are described, for instance, in Griffiths-Jones, Nucleic Acids Research, 2004, 32; or Griffiths-Jones et al., Nucleic Acids Research, 2008, 36. The sequences for particular microRNAs, including human sequences, are listed for instance in the specific Database (http://microrna.sangenac.uk).

WO2009/033185 relates to the identification of microRNAs that are characteristic of a subject infected by a virus. WO2010/109017 and WO2011/076142 relate to the identification of microRNAs that represent markers of cancer. WO2009/085234 proposes to use microRNAs as biomarkers of immunomodulatory drug activity. WO2011/025919 relates to the identification of microRNAs that represent potential biomarkers of lung disease.

Omoto et al (Retrovirology, vol. 1(1), 2004, 44) relates to the identification of a microRNA that can suppress expression of nef in HIV infected subjects. Houzet et al (Retrovirology, vol. 5(1), 2008, 118) indicates that microRNAs can be used to detect the presence of HIV in a subject. Peng et al (PLOS ONE Vol 6(12), 2011, e28486) relates to microRNAs characteristic of hepatocellular carcinoma in Hepatitis B infected subjects. Witwer et al (AIDS vol 25(17), 2011, 2057) discloses a microRNA signature of acute lentiviral infection and the use thereof as a biomarker of CNS disease. Lunbiao et al (PLOS ONE Vol 6(11), 2011, e27071) relates to microRNA that discriminate between an enterovirus and a coxsackievirus.

None of these references discloses microRNA that can discriminate viruses based on their tropism. More specifically, none of these references discloses methods for determining co-receptor usage of a virus in a subject.

In WO2011/027075, a method has been described for the identification of the tropism of a virus based on cellular microRNAs. According to this technique, a sample of a virus from a subject is contacted with a test cell (in particular Jurkatt cells), and the expression of microRNAs in said test cell is analyzed. There is no direct measure of microRNA levels in a sample from a patient disclosed in this document.

### SUMMARY OF THE INVENTION

The present invention relates to a method for analyzing the tropism of a virus based on circulating microRNAs. More specifically, the invention stems from the discovery that circulating microRNAs are present at sufficient levels in biological fluids of subjects infected by a virus, which provide an indication of the viral tropism in said subject. The invention therefore allows a direct measure in a biological fluid, which is simple and more cost effective.

An object of this invention relates to an in vitro method for determining the tropism of a virus in an infected subject, comprising a determination of the presence or level of at least one circulating microRNA in a biological sample from the subject, and correlating said determination to the tropism of the virus.

In a particular embodiment, the method comprises determining a circulating microRNA profile from said sample and comparing said profile to at least one reference profile characteristic of a virus tropism, wherein a substantial similarity in said profiles indicates the tropism of the virus in the sample.

A further object of this invention relates to an in vitro method for determining the tropism of a virus in an infected subject, comprising a determination of the presence or level of at least one PBMC microRNA in a biological sample from the subject, and correlating said determination to the tropism of the virus.

The virus in the subject may be any virus that infects mammalian cells, preferably any virus that infects human beings. In a preferred embodiment, the virus is human immunodeficiency virus (HIV).

In this regard, in a particular embodiment, the invention provides a method for determining whether HIV in a subject uses the CXCR4 or the CCR5 co-receptor. Such a determination is particularly relevant since the treatment differs depending on HIV tropism.

A specific object of the invention also relates to a method for determining whether HIV in a subject uses the CXCR4 or the CCR5 co-receptor, comprising determining in a test sample from the subject, typically in a plasma sample, more particularly in a platelet-poor plasma sample, the circulating level of at least one microRNA selected from hsa-let-7f-2#; hsa-miR-106a; hsa-miR-1208; hsa-miR-1233; hsa-miR-1243; hsa-miR-1262; hsa-miR-1267; hsa-miR-1276; hsa-miR-1285; hsa-miR-1290; hsa-miR-1298; hsa-miR-1305; hsa-miR-140-3p; hsa-miR-142-3p; hsa-miR-144#; hsa-miR-146a; hsa-miR-150; hsa-miR-17; hsa-miR-186; hsa-miR-20a; hsa-miR-210; hsa-miR-222; hsa-miR-223; hsa-miR-24; hsa-miR-24-2#; hsa-miR-26b; hsa-miR-30a-3p; hsa-miR-30a-5p; hsa-miR-30c; hsa-miR-320; hsa-miR-323-3p; hsa-miR-338-5P; hsa-miR-33a; hsa-miR-33b; hsa-miR-340; hsa-miR-342-3p; hsa-miR-378; hsa-miR-424; hsa-miR-483-5p; hsa-miR-484; hsa-miR-485-3p; hsa-miR-486; hsa-miR-486-3p; hsa-miR-502; hsa-miR-550; hsa-miR-557; hsa-miR-572; hsa-miR-575; hsa-miR-581; hsa-miR-582-3p; hsa-miR-584; hsa-miR-586; hsa-miR-596; hsa-miR-597; hsa-miR-625#; hsa-miR-630; hsa-miR-638; hsa-miR-645; hsa-miR-648; hsa-miR-656; hsa-miR-657; hsa-miR-659; hsa-miR-661; hsa-miR-720; hsa-miR-770-5p; hsa-miR-875-5p; hsa-miR-892b; hsa-miR-99b#; mmu-miR-451; or mmu-miR-93; the circulating level of each of said microRNAs being correlated to CXCR4 or CCR5 receptor usage by HIV.

A specific object of the invention also relates to a method for determining whether HIV in a subject uses the CXCR4 or the CCR5 co-receptor, comprising determining in a PBMC sample from the subject the level of at least one microRNA selected from hsa-let-7a; hsa-let-7d; hsa-let-7e; hsa-let-7f; hsa-let-7g; hsa-miR-103; hsa-miR-106a; hsa-miR-106b; hsa-miR-124-3p (mmu-miR-124a); hsa-miR-1244; hsa-miR-126; hsa-miR-126#; hsa-miR-1260; hsa-miR-1274A; hsa-miR-1276; hsa-miR-130a; hsa-miR-130b; hsa-miR-133a; hsa-miR-134 (mmu-miR-134); hsa-miR-139-5p; hsa-miR-140-5p (mmu-miR-140); hsa-miR-142-3p; hsa-miR-142-5p; hsa-miR-146a; hsa-miR-146b; hsa-miR-148a; hsa-miR-148b; hsa-miR-149#; hsa-miR-150; hsa-miR-151-5P; hsa-miR-15b; hsa-miR-16; hsa-miR-17; hsa-miR-1825; hsa-miR-185; hsa-miR-186; hsa-miR-18b; hsa-miR-191; hsa-miR-192; hsa-miR-195; hsa-miR-196b; hsa-miR-199a-3p; hsa-miR-19a; hsa-miR-19b; hsa-miR-200b; hsa-miR-20a; hsa-miR-20b; hsa-miR-21; hsa-miR-221; hsa-miR-223; hsa-miR-223#; hsa-miR-24-2#; hsa-miR-25; hsa-miR-26a; hsa-miR-26b; hsa-miR-27a; hsa-miR-27a#; hsa-miR-28; hsa-miR-299-3p; hsa-miR-29a; hsa-miR-29c; hsa-miR-301; hsa-miR-301b; hsa-miR-30b; hsa-miR-30c; hsa-miR-31; hsa-miR-324-3p; hsa-miR-328; hsa-miR-335; hsa-miR-340; hsa-miR-340#; hsa-miR-342-3p; hsa-miR-365; hsa-miR-374; hsa-miR-374b-5p (mmu-miR-374-5p); hsa-miR-376c; hsa-miR-380-5p; hsa-miR-410; hsa-miR-422a; hsa-miR-425-5p; hsa-miR-432; hsa-miR-451a (mmu-miR-451); hsa-miR-454; hsa-miR-486; hsa-miR-494; hsa-miR-495 (mmu-miR-495); hsa-miR-516-3p; hsa-miR-522; hsa-miR-532; hsa-miR-532-3p; hsa-miR-543; hsa-miR-571; hsa-miR-574-3p; hsa-miR-590-3P; hsa-miR-590-5p; hsa-miR-628-5p; hsa-miR-638; hsa-miR-642; hsa-miR-650; hsa-miR-651; hsa-miR-652; hsa-miR-660; hsa-miR-7-1-3p (rno-miR-7#); hsa-miR-744#; hsa-miR-765; hsa-miR-875-5p; hsa-miR-93-5p (mmu-miR-93); or hsa-miR-942; the level of each of said microRNAs being correlated to CXCR4 or CCR5 receptor usage by HIV.

A further object of the invention resides in a microarray comprising a nucleic acid probe specific for at least one circulating microRNA characteristic of a virus tropism. Preferably, the microarray comprises a plurality of nucleic acid probes, said plurality of probes comprising probes specific for each circulating microRNAs of a microRNA profile characteristic of a virus tropism.

Another aspect of the invention relates to a set of nucleic acid primers comprising a plurality of nucleic acid primers, said plurality comprising primers that specifically amplify each circulating microRNA of a microRNA profile characteristic of a virus tropism.

The present invention also relates to a method for treating a subject infected by a virus, the method comprising determining the tropism of the virus infecting said subject by a method as disclosed above, and treating the subject with a therapy adapted to the virus tropism.

The invention also relates to the use of circulating microRNAs to determine the tropism of a virus.

### BRIEF DESCRIPTION OF THE DRAWINGS

FIG. 1: Analysis of circulating microRNAs in sera using the Qiagen kit (high panel) or the Macherey-Nagel kit (low panel).

FIG. 2: Analysis of circulating microRNAs in saliva using the Macherey-Nagel (FIG. 2, left panel) or the Norgen kit (FIG. 2, right panel), respectively. Analysis was carried out on Agilent 6000 (FIG. 2A) or Agilent Small RNA (FIG. 2B).

FIG. 3: Detection of microRNAs in circulating PBMCs. (A): RNA extracted using the Macherey-Nagel kit. Analysis on Nano 6000 chip, (left pattern) and Small RNA chip (right panel); (B): RNA extracted using the Ambion kit. Analysis on Nano 6000 chip, (left pattern) and Small RNA chip (right panel); (C): RNA extracted using the Qiagen kit. Analysis on Nano 6000 chip, (left pattern) and Small RNA chip (right panel); (D): RNA extracted using the Qiagen kit. Analysis on the Small RNA chip.

FIG. 4: Quantitative RT-PCR (qRT-PCR) of synthetic miRNA-638.

FIG. 5: Identification of miRNA-638 in serums of donors.

FIG. 6: Circulating MiRNA-638 (μg/μL) in PBMC and serum.

FIG. 7: Circulating MiRNA-638 (μg/μL) in saliva.

FIG. 8: miRNA amplifications using TaqMan qPCR array on PBMC samples (as example) from HIV patients infected by R5, X4 or Dual HIV strains and from healthy donors (Control samples).

FIG. 9: Relative quantification of miRNA in PPP and PBMC samples of HIV groups compared to the control group (from minimun 10 patients/group). Each bar represents 1 miRNA.

FIG. 10: ROC curves of tested miRNA combination in PPP and PBMC for group discrimination.

### DETAILED DESCRIPTION OF THE INVENTION

The present invention relates to a method for analyzing the tropism of a virus based on circulating microRNAs. More specifically, the invention stems from the discovery that circulating microRNAs are present at sufficient levels in biological fluids of subjects infected by a virus, which provide an indication of the viral tropism in said subject. The invention therefore allows a direct measure in a biological fluid, which is simple and more cost effective.

Within the context of the invention, the viral “tropism” designates (i) co-receptor usage by a virus and/or (ii) the cell type that are infected by a virus. It is known that a number of viruses use co-receptors for cell entry. For instance, the HIV interacts with target cells through the CD4 protein and also through a second co-receptor. The main co-receptors are CXCR4 and CCR5. The co-receptor usage by the HIV reflects the progression of the immunodeficiency. HIV viruses that use CXCR4 are usually associated with the AIDS disease. Examples of further co-receptors used by HIV are CCR1, CCR2, CCR3, CCR4, CCR8, CCR9, CXCR2, or STRL33. Further information regarding these co-receptors is contained in WO2011/027075. Determining the viral tropism of a virus therefore includes, in a specific embodiment, determining co-receptor usage of a virus. In relation to the HIV, this includes more preferably determining the presence of a virus that uses CXCR4. As mentioned above, the viral tropism also designates the analysis of cell types infected by a virus. The invention also allows the discrimination, between viruses of a same family, of a virus that has tropism for a certain cell type.

A preferred embodiment of the invention resides in a method for determining co-receptor usage by a virus.

A specific and most preferred embodiment of the invention is a method for identifying HIV strains that use CXCR4.

The invention is based on a measure or dosage of circulating microRNAs. Within the context of this invention the term “microRNA” is meant to include mature single stranded microRNAs, as well as precursors and variants thereof, which may be naturally occurring. The term “microRNA” may also include primary (pri-miRNA) and precursor (pre-miRNA) microRNA transcripts and duplex miRNAs. In a typical embodiment, the invention comprises a determination of mature microRNAs. As an example, the designation miR-638 refers to a mature microRNA sequence derived from pre-miR-638.

A “circulating” microRNA is a microRNA that is present outside of a cell, particularly in a biological fluid in an organism. A circulating level of a microRNA is the level or concentration of that microRNA in a fluid. The invention is based on a direct measure of circulating microRNAs in biological samples, preferably without any treatment to release cellular microRNAs that are contained in cells. In this regard, in a preferred embodiment, the present invention comprises the detection of circulating microRNAs in biological samples that are essentially devoid of cells, or that are treated to remove cells. Preferred examples of biological samples include samples of biological fluids.

MicroRNA levels in circulating PBMCs designates the amount or concentration of a specified microRNA detected in PBMCs obtained from a fluid sample of a subject.

Methods for Determining Circulating microRNAs

Detection of a microRNA according to the invention includes detecting the presence of such microRNA or, preferably, measuring the (relative or absolute) amount of a microRNA. Methods for detecting or measuring the amount of microRNAs are known per se in the art. Such methods include, without limitation, quantitative reverse transcriptase polymerase chain reaction (RT-PCR), hybridization with specific probes, northern blot, affinity binding, or the like.

In a particular embodiment, microRNAs are detected or measured in the sample by hybridization, amplification, ligand-binding, or a functional assay. In a particular embodiment, an aliquot of the biological sample is contacted with a nucleic acid probe, a nucleic acid primer, or a ligand, characteristic of at least one target microRNA, and the presence or amount of complexes formed between said probe, primer, or ligand and nucleic acids in the aliquot is determined. In such methods, the probe or ligand may be in solution or immobilized on a support, such as a microarray. Also, the biological sample may be treated prior to determination, e.g., diluted or concentrated or enriched for microRNAs. The microRNAs in the sample may also be labeled to facilitate determination.

In a particular embodiment, the method comprises (i) optionally labeling circulating microRNAs present in a test biological sample, and (ii) hybridizing said (optionally labeled) microRNAs to a support (such as a microarray) comprising at least one nucleic acid probe specific for a microRNA characteristic of a virus tropism to obtain a hybridization profile, the hybridization profile being indicative of the tropism of the virus in the infected subject.

In another particular embodiment, the method comprises (i) obtaining circulating microRNAs from a test biological sample, and (ii) contacting said circulating microRNAs with a set of at least two, preferably at least three nucleic acid primers, said at least two, preferably at least three nucleic acid primers specifically amplifying a microRNA characteristic of a virus tropism, to obtain an amplification product, the amplification product being indicative of the tropism of the virus in the infected subject.

In a preferred embodiment, circulating microRNAs are determined by microarray analysis, i.e., by contacting a test sample with a microarray comprising a plurality of specific probes immobilized on its surface, allowing hybridization reaction to occur, and analyzing the hybridization profile.

To analyze circulating microRNAs, the first step is to obtain and/or prepare the biological sample. The test sample may be obtained from any biological sample that contains circulating microRNAs or circulating PBMCs. In a particular embodiment, the test sample is or is obtained from a biological fluid, such as without limitation blood, plasma, serum, saliva, urine, cerebrospinal fluid, bronchial lavage fluid or lavage fluid from sinus. Other fluids may also be used to perform the invention such as bone marrow, cervical, vaginal, uretral, anal, throat, gingival, or ocular swab, lymph, aqueous humor, amniotic fluid, cerumen, breast milk, semen, prostatic fluid, female ejaculate, sweat, tears, cyst fluid, pleural or peritoneal fluid, pericardial fluid, interstitial fluid, menses, pus, sebum, vaginal secretions, mucosal secretion, bronchopulmonary aspirates, or umbilical cord blood.

Various methods exist for obtaining and preparing biological fluids such as blood, serum, or plasma samples. In addition, blood collection tubes are commercially available from many sources. A preferred sample is blood, or plasma, such as platelet-poor plasma.

Preferably, for analyzing circulating microRNAs, the test sample is collected and processed within 1-24 hours to minimize degradation of circulating microRNAs and to minimize the release of microRNAs from intact cells in the sample. The test sample (e.g., blood, plasma, serum, urine, saliva, and others) may be frozen and processed later.

For analyzing microRNAs in circulating PBMCs, the test sample is collected, treated to separate cells (e.g., by centrifugation), and treated to release microRNAs from circulating PBMCs. The sample is preferably processed within 1-24 hours after release of the microRNAs from circulating PBMCs to minimize degradation of circulating microRNAs. The test sample (e.g., blood, plasma, serum, saliva) may be frozen and processed later.

Preferably, prior to determining circulating microRNA levels, the test sample is treated. Treatment may be performed to normalize microRNAs, dilute or concentrate the sample, enrich for microRNAs, label microRNAs, remove cells or other fractions, protect RNA (e.g., inhibit RNase), etc.

Furthermore, our results also show that the sample may be frozen and subsequently thawed, without altering the reliability of the dosage. Accordingly, in a particular embodiment, the method comprises:
a) Providing a biological sample collected from a subject, the sample comprising circulating microRNAs,
b) Treating the sample by dilution, concentration, enrichment in microRNAs, protection of RNAs, or for removing cells,
c) Optionally freezing the sample, and
d) Determining circulating microRNAs in the sample, preferably less than 48 hours after collection or thawing of the sample, more preferably less than 24, 18, 12 or 6 hours, typically between 1-6 hours.

Steps b) and c) may be inverted. In such a case, the sample is thawed prior to the treatment step.

Circulating microRNAs may be extracted or partially purified from the biological sample prior to determination. To that purpose, total circulating RNAs may be purified by homogenization in the presence of a nucleic acid extraction buffer, followed by centrifugation. RNA molecules may be separated by electrophoresis on agarose gel(s) following standard techniques. Kits are commercially available to prepare microRNAs from biological samples.

For determining a selected microRNA by hybridization, one or several suitable probes specific for said given microRNA can be produced using the nucleotide sequence of said microRNA. The nucleic acid sequences of microRNAs are available from the “miRBase::Sequences” database of the Wellcome Trust Sanger Institute (http://microrna.sangetac.uk/sequences/index.shtml). The nucleic acid sequences of all microRNAs identified in the present invention are listed in Tables I and IV. Preferred probes are single stranded nucleic acid molecules of 10-500 bases in length, more preferably 10-200. Most preferred probes have a length similar to that of the target microRNA. They contain a sequence that is complementary to the target microRNA, preferably a sequence that is perfectly complementary. In certain embodiments, a level of mismatch may be tolerated as long as the probe may specifically hybridize to the target microRNA in a complex sample, when placed under stringent conditions.

Methods for labeling DNA or RNA probes, and the conditions for hybridization thereof to target nucleic acids are known per se in the art, as described e.g., in Molecular Cloning: A Laboratory Manual, J. Sambrook et al., eds., 2nd edition, Cold Spring Harbor Laboratory Press, 1989, incorporated therein by reference.

The relative number of circulating microRNAs in a sample can also be determined by specific amplification. Various techniques exist for amplifying microRNA nucleic acid sequences, including without limitation reverse transcription (RT), polymerase chain reaction (PCR), real-time PCR (quantitative PCR (q-PCR)), nucleic acid sequence-base amplification, multiplex ligatable probe amplification, rolling circle amplification, or strand displacement amplification. In a preferred embodiment, the determination is made by reverse transcription of microRNAs, followed by amplification of the reverse-transcribed transcripts by polymerase chain reaction (RT-PCR). Amplification generally uses nucleic acid primers. A primer is generally about 15 to 40 nucleotides in length, single stranded, and contains a region that is complementary to a portion of the target microRNA. Generally, a pair of primers is used comprising: a forward primer, that can anneal to the target microRNA, and a reverse primer, that is designed to anneal to the complement of the reverse transcribed target microRNA.

Generally, the level of circulating microRNA measured is compared to a control or reference value to determine whether the level is reduced or elevated. The control or reference may be an external control, such as a circulating microRNA in a test sample from a subject known to be infected by a virus having a given tropism. The external control may be a microRNA from a non-serum sample or a known amount of a synthetic RNA. The control may be a pooled, average, or individual sample. The control or reference value may be a mean or average reference value for a given microRNA in a reference situation.

In some embodiments, it is desirable to simultaneously determine the expression level of a plurality of different circulating microRNAs in the test sample. In certain instances, it may even be desirable to determine the expression level of all known microRNAs. Assessing expression levels for several circulating microRNAs may be performed using microarray or biochips comprising a plurality of probes or using a combination of various primers. The use of microarray or primers has many advantages for microRNA detection. Indeed, several hundreds of microRNAs can be identified in a single sample at one time point. Moreover, a small amount of RNA is needed. Assessing expression levels for several circulating microRNAs may also be performed using specific stem-loop RT followed by quantitative PCRs, particularly low density RT-qPCR like TLDA (TaqMan® Low Density Arrays; Life Technologies). As disclosed in the experimental section, such method can be used efficiently to assess simultaneously a large number of microRNAs in a complex test sample. The method may therefore comprise the determination of at least 2, 3, 4, 5, 10, 15, 20, 30, 40, 50 or even more circulating microRNAs.

In this regard, the invention also relates to circulating (human) microRNAs or microRNA profiles characteristic of a virus tropism.

Circulating microRNAs Correlated to Virus Tropism

The inventors have identified specific circulating microRNAs correlated with co-receptor tropism of a virus, particularly HIV. These circulating microRNAs have been identified from plasma and include, more specifically: hsa-let-7f-2#; hsa-miR-106a; hsa-miR-1208; hsa-miR-1233; hsa-miR-1243; hsa-miR-1262; hsa-miR-1267; hsa-miR-1276; hsa-miR-1285; hsa-miR-1290; hsa-miR-1298; hsa-miR-1305; hsa-miR-140-3p; hsa-miR-142-3p; hsa-miR-144#; hsa-miR-146a; hsa-miR-150; hsa-miR-17; hsa-miR-186; hsa-miR-20a; hsa-miR-210; hsa-miR-222; hsa-miR-223; hsa-miR-24; hsa-miR-24-2#; hsa-miR-26b; hsa-miR-30a-3p; hsa-miR-30a-5p; hsa-miR-30c; hsa-miR-320; hsa-miR-323-3p; hsa-miR-338-5P; hsa-miR-33a; hsa-miR-33b; hsa-miR-340; hsa-miR-342-3p; hsa-miR-378; hsa-miR-424; hsa-miR-483-5p; hsa-miR-484; hsa-miR-485-3p; hsa-miR-486; hsa-miR-486-3p; hsa-miR-502; hsa-miR-550; hsa-miR-557; hsa-miR-572; hsa-miR-575; hsa-miR-581; hsa-miR-582-3p; hsa-miR-584; hsa-miR-586; hsa-miR-596; hsa-miR-597; hsa-miR-625#; hsa-miR-630; hsa-miR-638; hsa-miR-645; hsa-miR-648; hsa-miR-656; hsa-miR-657; hsa-miR-659; hsa-miR-661; hsa-miR-720; hsa-miR-770-5p; hsa-miR-875-5p; hsa-miR-892b; hsa-miR-99b#; mmu-miR-451; or mmu-miR-93.

A specific object of the invention therefore resides in a method for determining the co-receptor usage of a virus in a subject, comprising determining the circulating level of any one of the above microRNA in a fluid sample derived from said subject, said level being correlated to receptor usage. More preferably, the fluid sample is a plasma sample, such as a platelet-poor plasma sample.

A specific object of the invention also relates to a method for determining whether HIV in a subject uses the CXCR4 or the CCR5 co-receptor, comprising determining in a test sample from the subject the circulating level of at least one of the above microRNAs, the circulating level of each of said microRNAs being correlated to CXCR4 or CCR5 receptor usage by HIV.

As shown in the experimental section, the level of these microRNAs is modulated in fluids of subjects infected by a virus, depending on the co-receptor usage of that virus. These microRNAs therefore allow, alone or in combinations, the detection of co-receptor usage from samples obtained from infected subjects.

The nucleic acid sequence of each of these microRNAs is represented in Table I (see also the sequence listing).

Preferred circulating microRNAs for use in the invention are selected from hsa-miR-106a; hsa-miR-1208; hsa-miR-1233; hsa-miR-1267; hsa-miR-1290; hsa-miR-146a; hsa-miR-150; hsa-miR-17; hsa-miR-186; hsa-miR-20a; hsa-miR-222; hsa-miR-223; hsa-miR-24; hsa-miR-26b; hsa-miR-30a-5p; hsa-miR-30c; hsa-miR-323-3p; hsa-miR-338-5P; hsa-miR-33b; hsa-miR-342-3p; hsa-miR-378; hsa-miR-424; hsa-miR-483-5p; hsa-miR-484; hsa-miR-486; hsa-miR-486-3p; hsa-miR-572; hsa-miR-596; hsa-miR-625#; hsa-miR-638; hsa-miR-659; hsa-miR-661; hsa-miR-99b#; or mmu-miR-93.

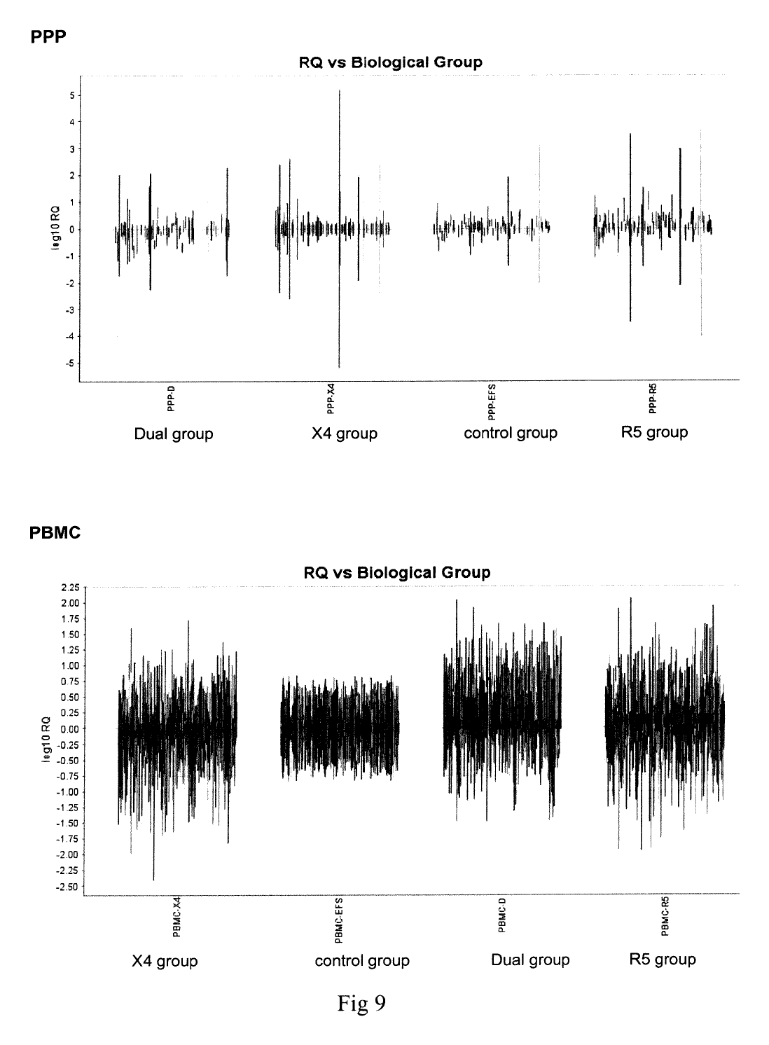

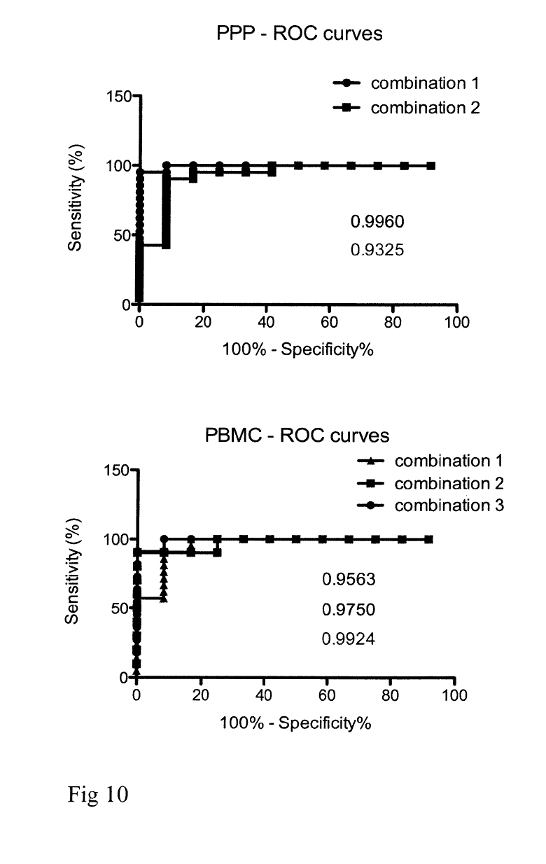

The modulation of each of these microRNAs is provided in Tables II and III, and in FIG. 9, as well as their correlation to co-receptor usage.

As can be inferred from this Table, the following circulating microRNAs are up-regulated in a fluid of a subject infected by HIV which uses CCR5 as a co-receptor: hsa-miR-146a, hsa-miR-661, hsa-miR-483-5p, hsa-miR-30a-5p, hsa-miR-222, hsa-miR-638, hsa-miR-572, hsa-miR-1208 (compared to control).

As can be inferred from this Table, the following circulating microRNAs are down-regulated in a fluid of a subject infected by HIV which uses CCR5 as a co-receptor: hsa-miR-486, hsa-miR-26b, hsa-miR-17, hsa-miR-106a, mmu-miR-93 (hsa-miR-93-5p), hsa-miR-20a (compared to control).

As can be inferred from this Table, the following circulating microRNAs are up-regulated in a fluid of a subject infected by HIV which uses CXCR4 as a co-receptor: hsa-miR-146a, hsa-miR-483-5p, hsa-miR-222, hsa-miR-150, hsa-miR-30c, hsa-miR-486, hsa-miR-484, hsa-miR-486-3p, hsa-miR-342-3p (compared to control and R5 groups).

As can be inferred from this Table, the following circulating microRNAs are down-regulated in a fluid of a subject infected by HIV which uses CXCR4 as a co-receptor: hsa-miR-661, hsa-miR-659, hsa-miR-30a-5p, hsa-miR-638, hsa-miR-625#, hsa-miR-572, hsa-miR-596 (compared to control and R5 groups).

In a particular embodiment, the invention relates to a method for detecting a circulating microRNA, the method comprising obtaining a test sample comprising circulating microRNAs, optionally treating the sample to remove cells, and assessing the presence or amount of at least one, typically at least 2, 3, 4, 5, 6, 7, 8, 9, or 10 distinct circulating microRNAs in said sample selected from hsa-let-7f-2#; hsa-miR-106a; hsa-miR-1208; hsa-miR-1233; hsa-miR-1243; hsa-miR-1262; hsa-miR-1267; hsa-miR-1276; hsa-miR-1285; hsa-miR-1290; hsa-miR-1298; hsa-miR-1305; hsa-miR-140-3p; hsa-miR-142-3p; hsa-miR-144#; hsa-miR-146a; hsa-miR-150; hsa-miR-17; hsa-miR-186; hsa-miR-20a; hsa-miR-210; hsa-miR-222; hsa-miR-223; hsa-miR-24; hsa-miR-24-2#; hsa-miR-26b; hsa-miR-30a-3p; hsa-miR-30a-5p; hsa-miR-30c; hsa-miR-320; hsa-miR-323-3p; hsa-miR-338-5P; hsa-miR-33a; hsa-miR-33b; hsa-miR-340; hsa-miR-342-3p; hsa-miR-378; hsa-miR-424; hsa-miR-483-5p; hsa-miR-484; hsa-miR-485-3p; hsa-miR-486; hsa-miR-486-3p; hsa-miR-502; hsa-miR-550; hsa-miR-557; hsa-miR-572; hsa-miR-575; hsa-miR-581; hsa-miR-582-3p; hsa-miR-584; hsa-miR-586; hsa-miR-596; hsa-miR-597; hsa-miR-625#; hsa-miR-630; hsa-miR-638; hsa-miR-645; hsa-miR-648; hsa-miR-656; hsa-miR-657; hsa-miR-659; hsa-miR-661; hsa-miR-720; hsa-miR-770-5p; hsa-miR-875-5p; hsa-miR-892b; hsa-miR-99b#; mmu-miR-451; or mmu-miR-93.

In a preferred embodiment, the invention relates to a method for detecting a circulating microRNA, the method comprising obtaining a test sample comprising circulating microRNAs, optionally treating the sample to remove cells, and assessing the presence or amount of at least one, typically at least 2, 3, 4, 5, 6, 7, 8, 9, or 10 distinct circulating microRNAs in said sample selected from hsa-miR-106a; hsa-miR-1208; hsa-miR-1233; hsa-miR-1267; hsa-miR-1290; hsa-miR-146a; hsa-miR-150; hsa-miR-17; hsa-miR-186; hsa-miR-20a; hsa-miR-222; hsa-miR-223; hsa-miR-24; hsa-miR-26b; hsa-miR-30a-5p; hsa-miR-30c; hsa-miR-323-3p; hsa-miR-338-5P; hsa-miR-33b; hsa-miR-342-3p; hsa-miR-378; hsa-miR-424; hsa-miR-483-5p; hsa-miR-484; hsa-miR-486; hsa-miR-486-3p; hsa-miR-572; hsa-miR-596; hsa-miR-625#; hsa-miR-638; hsa-miR-659; hsa-miR-661; hsa-miR-99b#; or mmu-miR-93.

In a particular embodiment, the invention comprises the detection of at least one circulating microRNA in a fluid of the subject selected from at least one the following categories, preferably at least one circulating microRNA from at least 2 of the following categories, even more preferably at least one circulating microRNA from each of the following categories:
at least one microRNA that is up-regulated in a fluid of a subject infected by a HIV which uses R5 as a co-receptor;
at least one microRNA that is down-regulated in a fluid of a subject infected by a HIV which uses R5 as a co-receptor;
at least one microRNA that is up-regulated in a fluid of a subject infected by a HIV which uses X4 as a co-receptor; and
at least one microRNA that is down-regulated in a fluid of a subject infected by a HIV which uses X4 as a co-receptor.

In this regard, preferred groups of microRNAs for detecting HIV co-receptor usage in a fluid (e.g., plasma) sample from a subject include the following microRNAs:

the following combinations of miRNAs in PPP may be able to discriminate X4/Dual-tropic HIV infected patients from R5-tropic HIV infected patients:
hsa-miR-661 and hsa-miR-638;
hsa-miR-30a-5p and hsa-miR-638,
hsa-miR-661 and hsa-miR-30a-5p,
hsa-miR-661, hsa-miR-638, and hsa-miR-30a-5p,
hsa-miR-661, hsa-miR-638, and hsa-miR-659, or
hsa-miR-661, hsa-miR-638, hsa-miR-30a-5p, hsa-miR-659, hsa-miR-596, hsa-miR-1233, hsa-miR-572, and hsa-miR-625#.

In this regard, preferred embodiments of the invention relate to methods for detecting HIV co-receptor usage in an infected subject, the method comprising determining the level of any one of the above combinations of microRNAs in a fluid sample from the subject.

In an alternative embodiment, the circulating microRNAs are selected from miRNA-638, hsa-miR-574-5p, hsa-miR-663, hsa-miR-149, hsa-miR-575, hsa-miR-638, hsa-miR-181b, hsa-let-7g, hsa-miR-30a, hsa-miR-148a, and hsa-miR-9.

In this regard, in a particular embodiment, the invention relates to a method for detecting a circulating microRNA, the method comprising obtaining a test sample comprising circulating microRNAs, optionally treating the sample to remove cells, and assessing the presence or amount of at least one, preferably at least 2, 3, 4, 5, 6, 7, 8, 9, or 10 distinct circulating microRNA in said sample selected from miRNA-638, hsa-miR-574-5p, hsa-miR-663, hsa-miR-149, hsa-miR-575, hsa-miR-638, hsa-miR-181b, hsa-let-7g, hsa-miR-30a, hsa-miR-148a, and hsa-miR-9.

As indicated above, the nucleic acid sequence of these microRNAs is available from miRBase and is also included in the present application. The nucleic acid sequence of miRNA-638 is provided in SEQ ID NO: 179 (RNA) and SEQ ID NO: 57 (DNA).

The invention also relates to a method of identifying or generating a microRNA or a microRNA profile characteristic of a virus tropism, comprising:
(i) generating a circulating microRNA population from a biological sample of a subject infected with a virus having a particular tropism, and
(ii) comparing said circulating microRNA population to a reference circulating microRNA population generated from a sample of a subject infected with a virus having a different tropism,
wherein the circulating microRNAs distinctive between said two populations define a circulating microRNA or microRNA profile characteristic of the virus tropism.

The invention further relates to a microarray comprising a nucleic acid probe specific for at least one circulating microRNA characteristic of a virus tropism, preferably a nucleic acid specific for at least one, 2, 3, 4, 5, 6, 7, 8, 9, or 10 circulating microRNAs selected from hsa-let-7f-2#; hsa-miR-106a; hsa-miR-1208; hsa-miR-1233; hsa-miR-1243; hsa-miR-1262; hsa-miR-1267; hsa-miR-1276; hsa-miR-1285; hsa-miR-1290; hsa-miR-1298; hsa-miR-1305; hsa-miR-140-3p; hsa-miR-142-3p; hsa-miR-144#; hsa-miR-146a; hsa-miR-150; hsa-miR-17; hsa-miR-186; hsa-miR-20a; hsa-miR-210; hsa-miR-222; hsa-miR-223; hsa-miR-24; hsa-miR-24-2#; hsa-miR-26b; hsa-miR-30a-3p; hsa-miR-30a-5p; hsa-miR-30c; hsa-miR-320; hsa-miR-323-3p; hsa-miR-338-5P; hsa-miR-33a; hsa-miR-33b; hsa-miR-340; hsa-miR-342-3p; hsa-miR-378; hsa-miR-424; hsa-miR-483-5p; hsa-miR-484; hsa-miR-485-3p; hsa-miR-486; hsa-miR-486-3p; hsa-miR-502; hsa-miR-550; hsa-miR-557; hsa-miR-572; hsa-miR-575; hsa-miR-581; hsa-miR-582-3p; hsa-miR-584; hsa-miR-586; hsa-miR-596; hsa-miR-597; hsa-miR-625#; hsa-miR-630; hsa-miR-638; hsa-miR-645; hsa-miR-648; hsa-miR-656; hsa-miR-657; hsa-miR-659; hsa-miR-661; hsa-miR-720; hsa-miR-770-5p; hsa-miR-875-5p; hsa-miR-892b; hsa-miR-99b#; mmu-miR-451; or mmu-miR-93.

In a particular embodiment, the microarray comprises a plurality of nucleic acid probes, said plurality of probes comprising probes specific for each circulating microRNA of a microRNA profile characteristic of a virus tropism, typically selected from the following groups:
hsa-miR-661 and hsa-miR-638;
hsa-miR-30a-5p and hsa-miR-638,
hsa-miR-661 and hsa-miR-30a-5p,
hsa-miR-661, hsa-miR-638, and hsa-miR-30a-5p,
hsa-miR-661, hsa-miR-638, and hsa-miR-659, or
hsa-miR-661, hsa-miR-638, hsa-miR-30a-5p, hsa-miR-659, hsa-miR-596, hsa-miR-1233, hsa-miR-572, and hsa-miR-625#.

In the microarray, the probes are preferably immobilized on a surface, typically in an ordered arrangement.

The invention further relates to a microarray comprising a nucleic acid probe specific for at least one circulating microRNA characteristic of a virus tropism, preferably a nucleic acid specific for at least one circulating microRNA selected from miRNA-638, hsa-miR-574-5p, hsa-miR-663, hsa-miR-149, hsa-miR-575, hsa-miR-638, hsa-miR-181b, hsa-let-7g, hsa-miR-30a, hsa-miR-148a, and hsa-miR-9. In a particular embodiment, the microarray comprises a plurality of nucleic acid probes, said plurality of probes comprising probes specific for each circulating microRNA of a microRNA profile characteristic of a virus tropism. In a further particular embodiment, the invention relates to a microarray comprising a probe specific for each of the following microRNAs: miRNA-638, hsa-miR-574-5p, hsa-miR-663, hsa-miR-149, hsa-miR-575, hsa-miR-638, hsa-miR-181b, hsa-let-7g, hsa-miR-30a, hsa-miR-148a, and hsa-miR-9. In the microarray, the probes are preferably immobilized on a surface, typically in an ordered arrangement.

The invention also relates to a composition comprising a plurality of nucleic acid primers, said plurality comprising at least one primer that specifically amplifies at least one circulating microRNA characteristic of a virus tropism, preferably selected from hsa-let-7f-2#; hsa-miR-106a; hsa-miR-1208; hsa-miR-1233; hsa-miR-1243; hsa-miR-1262; hsa-miR-1267; hsa-miR-1276; hsa-miR-1285; hsa-miR-1290; hsa-miR-1298; hsa-miR-1305; hsa-miR-140-3p; hsa-miR-142-3p; hsa-miR-144#; hsa-miR-146a; hsa-miR-150; hsa-miR-17; hsa-miR-186; hsa-miR-20a; hsa-miR-210; hsa-miR-222; hsa-miR-223; hsa-miR-24; hsa-miR-24-2#; hsa-miR-26b; hsa-miR-30a-3p; hsa-miR-30a-5p; hsa-miR-30c; hsa-miR-320; hsa-miR-323-3p; hsa-miR-338-5P; hsa-miR-33a; hsa-miR-33b; hsa-miR-340; hsa-miR-342-3p; hsa-miR-378; hsa-miR-424; hsa-miR-483-5p; hsa-miR-484; hsa-miR-485-3p; hsa-miR-486; hsa-miR-486-3p; hsa-miR-502; hsa-miR-550; hsa-miR-557; hsa-miR-572; hsa-miR-575; hsa-miR-581; hsa-miR-582-3p; hsa-miR-584; hsa-miR-586; hsa-miR-596; hsa-miR-597; hsa-miR-625#; hsa-miR-630; hsa-miR-638; hsa-miR-645; hsa-miR-648; hsa-miR-656; hsa-miR-657; hsa-miR-659; hsa-miR-661; hsa-miR-720; hsa-miR-770-5p; hsa-miR-875-5p; hsa-miR-892b; hsa-miR-99b#; mmu-miR-451; or mmu-miR-93.

In a particular embodiment, the composition comprises a plurality of primers that specifically amplify at least two, even more preferably at least 3, 4, 5, 6, 7, 8, 9 or 10 circulating microRNAs characteristic of a virus tropism, preferably selected from:
hsa-miR-661 and hsa-miR-638;
hsa-miR-30a-5p and hsa-miR-638,
hsa-miR-661 and hsa-miR-30a-5p,
hsa-miR-661, hsa-miR-638, and hsa-miR-30a-5p,
hsa-miR-661, hsa-miR-638, and hsa-miR-659, or
hsa-miR-661, hsa-miR-638, hsa-miR-30a-5p, hsa-miR-659, hsa-miR-596, hsa-miR-1233, hsa-miR-572, and hsa-miR-625#.

The invention also relates to a composition comprising a plurality of nucleic acid primers, said plurality comprising at least one primer that specifically amplify at least one circulating microRNAs characteristic of a virus tropism, preferably selected from miRNA-638, hsa-miR-574-5p, hsa-miR-663, hsa-miR-149, hsa-miR-575, hsa-miR-638, hsa-miR-181b, hsa-let-7g, hsa-miR-30a, hsa-miR-148a, and hsa-miR-9. In a particular embodiment, the composition comprises a plurality of primers that specifically amplify at least one, preferably at least two, even more preferably at least 3, 4, 5, 6, 7, 8, 9 or 10 circulating microRNAs characteristic of a virus tropism, preferably selected from miRNA-638, hsa-miR-574-5p, hsa-miR-663, hsa-miR-149, hsa-miR-575, hsa-miR-638, hsa-miR-181b, hsa-let-7g, hsa-miR-30a, hsa-miR-148a, and hsa-miR-9.

The invention also relates to a composition or set of nucleic acid primers comprising a plurality of nucleic acid primers, said plurality comprising primers that specifically amplify each circulating microRNAs of a microRNA profile characteristic of a virus tropism.

The microRNA profile is obtainable by a method comprising generating a circulating microRNA population from a biological sample of a subject infected with a virus having a particular tropism and comparing said circulating microRNA population to a reference circulating microRNA population generated from a sample of a subject infected with a virus having a different tropism, wherein the circulating microRNAs distinctive between said two populations define the circulating microRNA profile characteristic of the virus tropism.

A specific object of the invention also relates to a method for determining whether HIV in a subject uses the CXCR4 or the CCR5 co-receptor, comprising determining in a test sample from the subject the circulating level of at least one microRNA selected from miRNA-638, hsa-miR-574-5p, hsa-miR-663, hsa-miR-149, hsa-miR-575, hsa-miR-638, hsa-miR-181b, hsa-let-7g, hsa-miR-30a, hsa-miR-148a, and hsa-miR-9, the circulating level of each of said microRNAs being correlated to CXCR4 or CCR5 receptor usage by HIV.

In a particular embodiment, the invention relates to a method for determining whether HIV in a subject uses the CXCR4 or the CCR5 co-receptor, comprising determining the circulating level of circulating miR-638 in a test sample from the infected subject and comparing said level to a reference level, an increase in said level being indicative of CXCR4 co-receptor usage by the virus.

The present invention further relates to a kit containing a microarray as defined above. The kit may further contain reagents for a hybridization or amplification reaction, and/or a control sample, and/or a manual of instructions, and the like.

The invention also relates to the use of a microRNA, microarray, primer or kit as defined above for determining in vitro the tropism of a virus in a subject.

Circulating PBMC microRNAs Correlated to Virus Tropism

In a particular aspect, the invention also relates to a method of determining the tropism of a virus in a subject by determining the presence or amount of microRNAs in circulating blood cells, particularly in circulating PBMCs, granulocytes, platelets and/or red cells, more particularly in circulating PBMCs. In a particular embodiment, the invention therefore comprises a step of isolating such circulating cells from the test sample and assessing the amount or presence of microRNAs in said cells.

In this regard, the inventors have identified the following microRNAs in circulating PBMCs that are correlated to the tropism of a virus, particularly HIV: hsa-let-7a; hsa-let-7d; hsa-let-7e; hsa-let-7f; hsa-let-7g; hsa-miR-103; hsa-miR-106a; hsa-miR-106b; hsa-miR-124-3p (mmu-miR-124a); hsa-miR-1244; hsa-miR-126; hsa-miR-126#; hsa-miR-1260; hsa-miR-1274A; hsa-miR-1276; hsa-miR-130a; hsa-miR-130b; hsa-miR-133a; hsa-miR-134 (mmu-miR-134); hsa-miR-139-5p; hsa-miR-140-5p (mmu-miR-140); hsa-miR-142-3p; hsa-miR-142-5p; hsa-miR-146a; hsa-miR-146b; hsa-miR-148a; hsa-miR-148b; hsa-miR-149#; hsa-miR-150; hsa-miR-151-5P; hsa-miR-15b; hsa-miR-16; hsa-miR-17; hsa-miR-1825; hsa-miR-185; hsa-miR-186; hsa-miR-18b; hsa-miR-191; hsa-miR-192; hsa-miR-195; hsa-miR-196b; hsa-miR-199a-3p; hsa-miR-19a; hsa-miR-19b; hsa-miR-200b; hsa-miR-20a; hsa-miR-20b; hsa-miR-21; hsa-miR-221; hsa-miR-223; hsa-miR-223#; hsa-miR-24-2#; hsa-miR-25; hsa-miR-26a; hsa-miR-26b; hsa-miR-27a; hsa-miR-27a#; hsa-miR-28; hsa-miR-299-3p; hsa-miR-29a; hsa-miR-29c; hsa-miR-301; hsa-miR-301b; hsa-miR-30b; hsa-miR-30c; hsa-miR-31; hsa-miR-324-3p; hsa-miR-328; hsa-miR-335; hsa-miR-340; hsa-miR-340#; hsa-miR-342-3p; hsa-miR-365; hsa-miR-374; hsa-miR-374b-5p (mmu-miR-374-5p); hsa-miR-376c; hsa-miR-380-5p; hsa-miR-410; hsa-miR-422a; hsa-miR-425-5p; hsa-miR-432; hsa-miR-451a (mmu-miR-451); hsa-miR-454; hsa-miR-486; hsa-miR-494; hsa-miR-495 (mmu-miR-495); hsa-miR-516-3p; hsa-miR-522; hsa-miR-532; hsa-miR-532-3p; hsa-miR-543; hsa-miR-571; hsa-miR-574-3p; hsa-miR-590-3P; hsa-miR-590-5p; hsa-miR-628-5p; hsa-miR-638; hsa-miR-642; hsa-miR-650; hsa-miR-651; hsa-miR-652; hsa-miR-660; hsa-miR-7-1-3p (rno-miR-7#); hsa-miR-744#; hsa-miR-765; hsa-miR-875-5p; hsa-miR-93-5p (mmu-miR-93); or hsa-miR-942.

As shown in the experimental section, the level of these microRNAs is modulated in circulating PBMCs of subjects infected by a virus, depending on the co-receptor usage of that virus. These microRNAs therefore allow, alone or in combinations, the detection of co-receptor usage from samples obtained from infected subjects.

The nucleic acid sequence of each of these microRNAs is represented in Table IV.

Preferred PBMC microRNAs for use in the invention are selected from hsa-let-7a; hsa-let-7e; hsa-miR-124-3p (mmu-miR-124a); hsa-miR-126; hsa-miR-126#; hsa-miR-130a; hsa-miR-146b; hsa-miR-150; hsa-miR-17; hsa-miR-186; hsa-miR-191; hsa-miR-199a-3p; hsa-miR-19b; hsa-miR-20a; hsa-miR-20b; hsa-miR-21; hsa-miR-221; hsa-miR-26b; hsa-miR-30b; hsa-miR-30c; hsa-miR-31; hsa-miR-374; hsa-miR-376c; hsa-miR-432; hsa-miR-451a (mmu-miR-451); hsa-miR-454; hsa-miR-486; hsa-miR-494; hsa-miR-495 (mmu-miR-495); hsa-miR-516-3p; hsa-miR-522; hsa-miR-574-3p; hsa-miR-7-1-3p (rno-miR-7#); or hsa-miR-875-5p.

The modulation of each of the microRNAs is provided in Tables V and VI, in FIG. 9, as well as their correlation to co-receptor usage.

As can be inferred from this Table, the following microRNAs are up-regulated in circulating PBMCs from subjects infected by a HIV which uses CCR5 as a co-receptor: mmu-miR-124a (hsa-miR-124-3p), hsa-miR-494, hsa-miR-875-5p (compared to control).

As can be inferred from this Table, the following microRNAs are down-regulated in circulating PBMCs from subjects infected by a HIV which uses CCR5 as a co-receptor:

hsa-miR-31, hsa-miR-374, hsa-miR-126, hsa-miR-376c, hsa-miR-126#, hsa-miR-186, hsa-miR-146b, hsa-miR-30c, hsa-miR-432, hsa-miR-150 (compared to control).

As can be inferred from this Table, the following microRNAs are up-regulated in circulating PBMCs from subjects infected by a HIV which uses CXCR4 as a co-receptor: mmu-miR-124a (hsa-miR-124-3p), mmu-miR-451, hsa-miR-486, hsa-miR-432, hsa-miR-150 (compared to control and R5 groups).

As can be inferred from this Table, the following microRNAs are down-regulated in circulating PBMCs from subjects infected by a HIV which uses CXCR4 as a co-receptor: hsa-miR-126, hsa-miR-376c, hsa-miR-126#, hsa-miR-221, hsa-miR-494, hsa-miR-875-5p, hsa-let-7a, hsa-miR-130a, hsa-miR-516-3p, hsa-miR-522, hsa-miR-574-3p (compared to control and R5 groups).

Preferred groups of PBMC microRNAs include the following:
hsa-miR-150 and hsa-miR-486 (this combination is particularly suited to discriminate X4/Dual-tropic HIV infected patients from R5-tropic HIV infected patients);
hsa-miR-150, hsa-miR-486, hsa-miR-522 and hsa-miR-574-3p (this combination is particularly suited to discriminate X4-tropic HIV infected patients from R5 tropic HIV infected patients).
hsa-miR-150, hsa-miR-486 and hsa-miR-522;
hsa-miR-150, hsa-miR-486 and hsa-miR-574-3p;
hsa-miR-126 and hsa-miR-146b;
hsa-miR-126, hsa-miR-146b, and hsa-miR-20a;
hsa-miR-126 and hsa-miR-20a;
hsa-miR-146b and (hsa-miR-20a or hsa-miR-21 or hsa-miR-376c or hsa-let-7e); or
hsa-miR-126, hsa-miR-146b, hsa-miR-20a, hsa-miR-21, hsa-miR-376c and hsa-let-7e (this combination is particularly suited to discriminate Dual tropic HIV infected patients from R5-tropic HIV infected patients).

In a particular embodiment, the invention relates to a method for detecting a microRNA, the method comprising obtaining a test sample comprising circulating PBMCs, treating the sample to release microRNAs, and assessing the presence or amount of at least one, typically at least 2, 3, 4, 5, 6, 7, 8, 9, or 10 distinct circulating microRNA in said sample selected from hsa-let-7a; hsa-let-7d; hsa-let-7e; hsa-let-7f; hsa-let-7g; hsa-miR-103; hsa-miR-106a; hsa-miR-106b; hsa-miR-124-3p (mmu-miR-124a); hsa-miR-1244; hsa-miR-126; hsa-miR-126#; hsa-miR-1260; hsa-miR-1274A; hsa-miR-1276; hsa-miR-130a; hsa-miR-130b; hsa-miR-133a; hsa-miR-134 (mmu-miR-134); hsa-miR-139-5p; hsa-miR-140-5p (mmu-miR-140); hsa-miR-142-3p; hsa-miR-142-5p; hsa-miR-146a; hsa-miR-146b; hsa-miR-148a; hsa-miR-148b; hsa-miR-149#; hsa-miR-150; hsa-miR-151-5P; hsa-miR-15b; hsa-miR-16; hsa-miR-17; hsa-miR-1825; hsa-miR-185; hsa-miR-186; hsa-miR-18b; hsa-miR-191; hsa-miR-192; hsa-miR-195; hsa-miR-196b; hsa-miR-199a-3p; hsa-miR-19a; hsa-miR-19b; hsa-miR-200b; hsa-miR-20a; hsa-miR-20b; hsa-miR-21; hsa-miR-221; hsa-miR-223; hsa-miR-223#; hsa-miR-24-2#; hsa-miR-25; hsa-miR-26a; hsa-miR-26b; hsa-miR-27a; hsa-miR-27a#; hsa-miR-28; hsa-miR-299-3p; hsa-miR-29a; hsa-miR-29c; hsa-miR-301; hsa-miR-301b; hsa-miR-30b; hsa-miR-30c; hsa-miR-31; hsa-miR-324-3p; hsa-miR-328; hsa-miR-335; hsa-miR-340; hsa-miR-340#; hsa-miR-342-3p; hsa-miR-365; hsa-miR-374; hsa-miR-374b-5p (mmu-miR-374-5p); hsa-miR-376c; hsa-miR-380-5p; hsa-miR-410; hsa-miR-422a; hsa-miR-425-5p; hsa-miR-432; hsa-miR-451a (mmu-miR-451); hsa-miR-454; hsa-miR-486; hsa-miR-494; hsa-miR-495 (mmu-miR-495); hsa-miR-516-3p; hsa-miR-522; hsa-miR-532; hsa-miR-532-3p; hsa-miR-543; hsa-miR-571; hsa-miR-574-3p; hsa-miR-590-3P; hsa-miR-590-5p; hsa-miR-628-5p; hsa-miR-638; hsa-miR-642; hsa-miR-650; hsa-miR-651; hsa-miR-652; hsa-miR-660; hsa-miR-7-1-3p (rno-miR-7#); hsa-miR-744#; hsa-miR-765; hsa-miR-875-5p; hsa-miR-93-5p (mmu-miR-93); or hsa-miR-942.

In a particular embodiment, the invention relates to a method for detecting a microRNA, the method comprising obtaining a test sample comprising circulating PBMCs, treating the sample to release microRNAs, and assessing the presence or amount of at least one, typically at least 2, 3, 4, 5, 6, 7, 8, 9, or 10 distinct circulating microRNA in said sample selected from hsa-let-7a; hsa-let-7e; hsa-miR-124-3p (mmu-miR-124a); hsa-miR-126; hsa-miR-126#; hsa-miR-130a; hsa-miR-146b; hsa-miR-150; hsa-miR-17; hsa-miR-186; hsa-miR-191; hsa-miR-199a-3p; hsa-miR-19b; hsa-miR-20a; hsa-miR-20b; hsa-miR-21; hsa-miR-221; hsa-miR-26b; hsa-miR-30b; hsa-miR-30c; hsa-miR-31; hsa-miR-374; hsa-miR-376c; hsa-miR-432; hsa-miR-451a (mmu-miR-451); hsa-miR-454; hsa-miR-486; hsa-miR-494; hsa-miR-495 (mmu-miR-495); hsa-miR-516-3p; hsa-miR-522; hsa-miR-574-3p; hsa-miR-7-1-3p (rno-miR-7#); or hsa-miR-875-5p.

The invention further relates to a microarray comprising a nucleic acid probe specific for at least one microRNA selected from hsa-let-7a; hsa-let-7d; hsa-let-7e; hsa-let-7f; hsa-let-7g; hsa-miR-103; hsa-miR-106a; hsa-miR-106b; hsa-miR-124-3p (mmu-miR-124a); hsa-miR-1244; hsa-miR-126; hsa-miR-126#; hsa-miR-1260; hsa-miR-1274A; hsa-miR-1276; hsa-miR-130a; hsa-miR-130b; hsa-miR-133a; hsa-miR-134 (mmu-miR-134); hsa-miR-139-5p; hsa-miR-140-5p (mmu-miR-140); hsa-miR-142-3p; hsa-miR-142-5p; hsa-miR-146a; hsa-miR-146b; hsa-miR-148a; hsa-miR-148b; hsa-miR-149#; hsa-miR-150; hsa-miR-151-5P; hsa-miR-15b; hsa-miR-16; hsa-miR-17; hsa-miR-1825; hsa-miR-185; hsa-miR-186; hsa-miR-18b; hsa-miR-191; hsa-miR-192; hsa-miR-195; hsa-miR-196b; hsa-miR-199a-3p; hsa-miR-19a; hsa-miR-19b; hsa-miR-200b; hsa-miR-20a; hsa-miR-20b; hsa-miR-21; hsa-miR-221; hsa-miR-223; hsa-miR-223#; hsa-miR-24-2#; hsa-miR-25; hsa-miR-26a; hsa-miR-26b; hsa-miR-27a; hsa-miR-27a#; hsa-miR-28; hsa-miR-299-3p; hsa-miR-29a; hsa-miR-29c; hsa-miR-301; hsa-miR-301b; hsa-miR-30b; hsa-miR-30c; hsa-miR-31; hsa-miR-324-3p; hsa-miR-328; hsa-miR-335; hsa-miR-340; hsa-miR-340#; hsa-miR-342-3p; hsa-miR-365; hsa-miR-374; hsa-miR-374b-5p (mmu-miR-374-5p); hsa-miR-376c; hsa-miR-380-5p; hsa-miR-410; hsa-miR-422a; hsa-miR-425-5p; hsa-miR-432; hsa-miR-451a (mmu-miR-451); hsa-miR-454; hsa-miR-486; hsa-miR-494; hsa-miR-495 (mmu-miR-495); hsa-miR-516-3p; hsa-miR-522; hsa-miR-532; hsa-miR-532-3p; hsa-miR-543; hsa-miR-571; hsa-miR-574-3p; hsa-miR-590-3P; hsa-miR-590-5p; hsa-miR-628-5p; hsa-miR-638; hsa-miR-642; hsa-miR-650; hsa-miR-651; hsa-miR-652; hsa-miR-660; hsa-miR-7-1-3p (rno-miR-7#); hsa-miR-744#; hsa-miR-765; hsa-miR-875-5p; hsa-miR-93-5p (mmu-miR-93); or hsa-miR-942. In a particular embodiment, the microarray comprises a plurality of nucleic acid probes, said plurality of probes comprising probes specific several PBMC microRNAs characteristic of a virus tropism. In a further particular embodiment, the invention relates to a microarray comprising a probe specific for at least 2, 3, 4, 5, 6, 7, 8, 9, or 10 distinct microRNAs selected from: hsa-let-7a; hsa-let-7e; hsa-miR-124-3p (mmu-miR-124a); hsa-miR-126; hsa-miR-126#; hsa-miR-130a; hsa-miR-146b; hsa-miR-150; hsa-miR-17; hsa-miR-186; hsa-miR-191; hsa-miR-199a-3p; hsa-miR-19b; hsa-miR-20a; hsa-miR-20b; hsa-miR-21; hsa-miR-221; hsa-miR-26b; hsa-miR-30b; hsa-miR-30c; hsa-miR-31; hsa-miR-374; hsa-miR-376c; hsa-miR-432; hsa-miR-451a (mmu-miR-451); hsa-miR-454; hsa-miR-486; hsa-miR-494; hsa-miR-495 (mmu-miR-495); hsa-miR-516-3p; hsa-miR-522; hsa-miR-574-3p; hsa-miR-7-1-3p (rno-miR-7#); or hsa-miR-875-5p. In the microarray, the probes are preferably immobilized on a surface, typically in an ordered arrangement.

The invention also relates to a composition comprising a plurality of nucleic acid primers, said plurality comprising at least one primer that specifically amplify at least one microRNAs selected from hsa-let-7a; hsa-let-7d; hsa-let-7e; hsa-let-7f; hsa-let-7g; hsa-miR-103; hsa-miR-106a; hsa-miR-106b; hsa-miR-124-3p (mmu-miR-124a); hsa-miR-1244; hsa-miR-126; hsa-miR-126#; hsa-miR-1260; hsa-miR-1274A; hsa-miR-1276; hsa-miR-130a; hsa-miR-130b; hsa-miR-133a; hsa-miR-134 (mmu-miR-134); hsa-miR-139-5p; hsa-miR-140-5p (mmu-miR-140); hsa-miR-142-3p; hsa-miR-142-5p; hsa-miR-146a; hsa-miR-146b; hsa-miR-148a; hsa-miR-148b; hsa-miR-149#; hsa-miR-150; hsa-miR-151-5P; hsa-miR-15b; hsa-miR-16; hsa-miR-17; hsa-miR-1825; hsa-miR-185; hsa-miR-186; hsa-miR-18b; hsa-miR-191; hsa-miR-192; hsa-miR-195; hsa-miR-196b; hsa-miR-199a-3p; hsa-miR-19a; hsa-miR-19b; hsa-miR-200b; hsa-miR-20a; hsa-miR-20b; hsa-miR-21; hsa-miR-221; hsa-miR-223; hsa-miR-223#; hsa-miR-24-2#; hsa-miR-25; hsa-miR-26a; hsa-miR-26b; hsa-miR-27a; hsa-miR-27a#; hsa-miR-28; hsa-miR-299-3p; hsa-miR-29a; hsa-miR-29c; hsa-miR-301; hsa-miR-301b; hsa-miR-30b; hsa-miR-30c; hsa-miR-31; hsa-miR-324-3p; hsa-miR-328; hsa-miR-335; hsa-miR-340; hsa-miR-340#; hsa-miR-342-3p; hsa-miR-365; hsa-miR-374; hsa-miR-374b-5p (mmu-miR-374-5p); hsa-miR-376c; hsa-miR-380-5p; hsa-miR-410; hsa-miR-422a; hsa-miR-425-5p; hsa-miR-432; hsa-miR-451a (mmu-miR-451); hsa-miR-454; hsa-miR-486; hsa-miR-494; hsa-miR-495 (mmu-miR-495); hsa-miR-516-3p; hsa-miR-522; hsa-miR-532; hsa-miR-532-3p; hsa-miR-543; hsa-miR-571; hsa-miR-574-3p; hsa-miR-590-3P; hsa-miR-590-5p; hsa-miR-628-5p; hsa-miR-638; hsa-miR-642; hsa-miR-650; hsa-miR-651; hsa-miR-652; hsa-miR-660; hsa-miR-7-1-3p (rno-miR-7#); hsa-miR-744#; hsa-miR-765; hsa-miR-875-5p; hsa-miR-93-5p (mmu-miR-93); or hsa-miR-942. In a particular embodiment, the composition comprises a plurality of primers that specifically amplify at least one, preferably at least two, even more preferably at least 3, 4, 5, 6, 7, 8, 9 or 10 microRNAs preferably selected from hsa-let-7a; hsa-let-7e; hsa-miR-124-3p (mmu-miR-124a); hsa-miR-126; hsa-miR-126#; hsa-miR-130a; hsa-miR-146b; hsa-miR-150; hsa-miR-17; hsa-miR-186; hsa-miR-191; hsa-miR-199a-3p; hsa-miR-19b; hsa-miR-20a; hsa-miR-20b; hsa-miR-21; hsa-miR-221; hsa-miR-26b; hsa-miR-30b; hsa-miR-30c; hsa-miR-31; hsa-miR-374; hsa-miR-376c; hsa-miR-432; hsa-miR-451a (mmu-miR-451); hsa-miR-454; hsa-miR-486; hsa-miR-494; hsa-miR-495 (mmu-miR-495); hsa-miR-516-3p; hsa-miR-522; hsa-miR-574-3p; hsa-miR-7-1-3p (rno-miR-7#); or hsa-miR-875-5p.

The present invention further relates to a kit containing a microarray as defined above. The kit may further contain reagents for a hybridization or amplification reaction, and/or a control sample, and/or a manual of instructions, and the like.

The invention also relates to the use of a microRNA, microarray, primer or kit as defined above for determining in vitro the tropism of a virus in a subject.

### Viruses

The present invention can be used to determine the tropism of any virus. It is particularly suited to determine the tropism of viruses that infect eukaryotic cells, particularly mammalian cells (e.g., human cells, canine cells, cat cells, avian cells, or murine cells, for instance). The invention is particularly adapted to determine the tropism of viruses that infect human beings.

Examples of viruses that have specific tropisms include, without limitation, retroviruses, herpes viruses, adenoviruses, enteroviruses, reoviruses, papillomaviruses, picornaviruses, pox viruses, flaviviruses, etc. Specific examples for which a specific tropism may be identified according to the invention include the following viruses: Human Immunodeficiency Virus (HIV-1 and HIV-2), the hepatitis A, B or C virus, Delta hepatitis virus, occult B hepatitis virus, measle virus, herpes virus (including HSV-1, HSV2, HSV-6, EBV and CMV), papillomaviruses, rotavirus, parvovirus, influenza virus, parainfluenza virus, rhinovirus, coronavirus, poxvirus, rotavirus, HTLV-1, HTLV-2, dengue virus, West Nile virus, Yellow fever virus, varicella zoster virus, SRAS, respiratory sincytial virus, Chikungunya virus, or hemorragic fever viruses (Arenaviridae, Filoviridae, Bunyaviridae, Flaviviridae); rubella virus, mumps virus, polio virus.

The invention also provides, in another aspect, a method for identifying an antiviral agent, comprising testing a candidate drug on an organism and determining circulating microRNAs after treatment, wherein a drug that modifies viral tropism represents a candidate antiviral agent.

The invention also relates to a method for treating a subject infected by a virus, the method comprising determining the tropism of the virus infecting said subject by a method as described above, and treating the subject with a therapy adapted to the virus tropism.

Further aspects and advantages of the invention will be disclosed in the following experimental section, which should be considered as illustrative. The contents of all references, patents and published patent applications cited throughout this application are incorporated herein by reference.

### Examples

We herein describe methods for identifying the cellular tropism of a virus, especially the HIV (Human Immunodeficiency virus) by measuring circulating miRNAs in biological samples.

### 1. Biological Samples

### 1.1. Serum and Plasma

Blood collection tubes are commercially available from many sources and in a variety of formats (e.g., Becton Dickenson Vacutainer tubes-SST™, glass serum tubes, or plastic serum tubes).

Blood samples are obtained from voluntary blood donors collected at the Etablissement Français du Sang of Montpellier, or from human patients previously screened for HIV contamination.

Serum is the non-cellular portion of coagulated blood. Plasma also does not contain peripheral blood cells, but unlike serum, plasma contains clotting factors.

Four to six mL of blood are withdrawn from controls or patients by venipuncture into collection tubes using standard methods. It is allowed to clot for 4 to 6 hours at room temperature and then keep at 4-8° C. When using plasma, blood is collected into EDTA blood tubes (Sarstedt, Monovette EDTA K) containing 1.6 mg EDTA. The EDTA prevents coagulation. Tubes were inverted 8-10 times immediately after blood collection

The serum or the plasma is then separated from the cellular portion of the coagulated or EDTA_treated blood, respectively, by centrifugation. Centrifugation to prepare serum or plasma is usually performed at a speed of 500 to 1,000×g, 10 min. Plasma is freed from platelets using known methods, such as by further centrifugation at a speed of 500 to 1,000×g, for 10 minutes, at room temperature, followed by 15,000 g for 20 minutes at room temperature in Eppendorf tubes. Platelet-Poor Plasmas (PPP) were gently collected from the tubes without collecting platelets.

Serum and plasma (or PPP) were aliquoted in 2 mL microtubes and frozen at −80° C. until it was subjected to RNA isolation. Alternatively, microRNAs were extracted using the appropriate kits and frozen under aliquots at −80° C.

### 1.2. Peripheral Blood Cells

Peripheral blood mononuclear cells are components of peripheral blood that can be easily isolated from the blood sample. They were isolated from fresh blood by Ficoll gradient separation and counted.

Then, 1 to 3×10E6 cells were mixed with the indicated volume of the lysis buffer of the kit, following manufacturer instructions, to achieve lysis and inactivate endogenous RNAses.

Lysates were frozen at −80° C. until RNA purification. Alternatively, microRNAs were extracted using the appropriate kits and frozen under aliquots at −80° C.

### 1.3. Saliva

Saliva samples were collected from healthy individuals. 200 μL of whole saliva were immediately lysed and mi-RNAs were extracted using the appropriate kits and frozen under aliquots at −80° C.

2. Extraction of miRNA

Circulating (free) RNA is usually present as short fragments of less than 1000 nt, and includes free-circulating miRNA (21nt). RNA Purification Kits provide an efficient method for the purification of all sizes of these fragmented free-circulating RNAs from human plasma or serum.

RNA may be extracted from serum or plasma or other biological fluids (saliva, urine) and purified using methods known per se in the art. Many methods are known for isolating total RNA, or for specifically extracting small RNAs, including miRNAs. The RNA may be extracted using commercially available kits (e.g., Norgen, Ambion, Life Technology, Agilent, Sigma, Qiagen, Roche, Texagen, Macherey Nagel, Amresco, Epicentre, ZymoReserach, BioMobile).

### 2.1. Serum or Plasma

Total RNA was isolated from 200-300 microL of serum or plasma using the following extraction kits:
Qiagen: miRNeasy Mini Kit. The kit is use for the purification of total RNA, including small RNAs, from serum or plasma.
Ambion: mirVana™ PARIS™ Kit. The mirVana™ PARIS™ Kit was designed for isolation of both protein and RNA suitable for studies of small RNA expression, processing or function. RNA can be isolated using a procedure that combines the advantages of organic extraction and solid-phase extraction, while avoiding the disadvantages of both. High yields of ultra-pure RNA can be prepared in about 30 min. The high quality RNA recovered can be used in any application, including RT-PCR, RNA amplification, microarray analyses, solution hybridization assays, and blot hybridization
Macherey-Nagel: NucleoSpin® miRNA Plasma. The kit offers the unique feature to isolate small RNA and DNA from plasma without the need to resort to the cumbersome phenol/chloroform extraction or a time consuming proteinase digest.
Norgen: Total RNA Purification Kit, and Plasma/Serum Circulating RNA Purification Kit
1. Total RNA Purification Kit, provides a rapid method for the isolation and purification of total RNA from cultured animal cells, tissue samples, blood, plasma, serum, bacteria, yeast, fungi, plants and viruses. The kit purifies all sizes of RNA. The RNA is preferentially purified from other cellular components such as proteins, without the use of phenol or chloroform.
2. Plasma/Serum Circulating RNA Purification Kit. The kit provides a fast, reliable and simple procedure for isolating circulating RNA from various amounts of plasma/serum ranging from 1 mL to 5 mL.

RNAs, of which miRNAs, were extracted from the biological fluids according to the manufacturer protocols.

### 2.2. Saliva

Two hundred microliters (200 μL) of the supernatant saliva were used for RNA extraction. Saliva samples were extracted using the following extraction kits:
Qiagen: miRNeasy Mini Kit.
Macherey-Nagel:
1. NucleoSpin® miRNA Plasma.
2. NucleoSpin® miRNA
Norgen: Total RNA Purification Kit.

RNAs, of which miRNAs, were extracted from the biological fluids according to the manufacturer protocols.

### 2.3. PBMC

RNAs were isolated from 1.10⁶ peripheral blood mononuclear cells (PBMC) lysates using the following extraction kits:
Qiagen: miRNeasy Mini Kit.
Ambion: mirVana™ PARIS™ Kit.
Macherey-Nagel: NucleoSpin® miRNA. The kit is designed for the simultaneous isolation of small RNA (<200 nt), large RNA (>200 nt), and protein in three separate fractions from a large variety of sample materials
Norgen:Total RNA Purification Kit. The Norgen's Total RNA Purification Kit provides a rapid method for the isolation and purification of total RNA from cultured animal cells, tissue samples, blood, plasma, serum, bacteria, yeast, fungi, plants and viruses. The kit purifies all sizes of RNA, from large mRNA and ribosomal RNA down to microRNA (miRNA) and small interfering RNA (siRNA). The RNA is preferentially purified from other cellular components such as proteins, without the use of phenol or chloroform. The purified RNA is of the highest integrity, and can be used in a number of downstream applications including real time PCR, reverse transcription PCR, Northern blotting, Rnase protection and primer extension, and expression array assays.

RNAs were extracted from PBMCs according to the manufacturer protocols.

All mentioned kits were tested on healthy donor samples. Only Macherey-Nagel kits were used to perform RNA extractions from plasma (e.g., PPP) and PBMC from HIV-infected patients.

### 3. Quality Control of Extracted Mi-RNA

### 3.1. Quality Control Assessment Using the Nanodrop Technology

Total miRNAs were extracted using the commercial kits described above. Total RNA was quantified using ND-1000 nanodrop spectrophotometer (Fischer scientific). Quality control was performed by assessing the OD ration of 260/280 nm. A ratio of 1.8 to 2.2 is expected to validate the quality of the RNA preparation.

### 3.1.1. RNA Extracted in PBMCs and Serum

The Table below summarizes the concentration (μg/1.10⁶ PBMC or /mL of Serum) of total RNA, and the OD ratio 260/280 obtained in one example, using 10 samples from healthy blood donors. RNA were prepared and tested immediately or frozen at −80° C. and tested after unthawing.

| | Mean values (standard deviation) | | | | | | | |
|---|---|---|---|---|---|---|---|---|
| | PBMCs (μg/1 · 10⁶ cells) | | | | Serum (μg/mL) | | | |
| | Before | | After | | Before | | After | |
| | freezing | | freezing | | freezing | | freezing | |
| Qiagen | 0.96 | (0.15) | 1.12 | (0.22) | 0.95 | (0.41) | 1.16 | (0.49) |
| Norgen | 1.93 | (0.66) | | | 0.66 | (0.21) | | |
| Ambion | 3.7 | (1.18) | 3.37 | (0.71) | 10.7 | (5.59) | 10.16 | (5.0) |

| | | | | | | |
|---|---|---|---|---|---|---|
| Macherey - | 1.0 | (0.21) | 1.22 | (0.24) | / | / |
| cells | | | | | | |

| | | | | | | |
|---|---|---|---|---|---|---|
| Macherey - | / | / | 0.91 | (0.46) | 1.26 | (0.3) |
| serum/plasma | | | | | | |

From PBMCs, total RNA concentrations are in the range of 0.96 to 3.7 μg/1.10⁶ PBMC. Very close values are obtained with preparations obtained with the investigated kits.

Circulating RNA concentrations are in the range of 0.95 to 10.7 μg/mL after extraction with the Qiagen or Macherey-Nagel serum/plasma. The Ambion kit provides higher quantity of RNA, as measured using an OD value.

Remarkably, no significant difference is obtained with frozen RNA or unfrozen RNA.

### 3.1.2. RNA Extracted in Saliva

The Table below summarizes the concentration (μg/mL of Saliva) of total RNA, and the OD ratio 260/280 obtained in one example, using 10 samples from healthy blood donors. RNA were prepared and tested immediately.

| Mean values (standard deviation) | | |
|---|---|---|
| | Concentration | Ratio |
| | (μg/mL) | (OD260/OD280) |

| | | | | |
|---|---|---|---|---|
| Norgen | 40.7 | (32.70) | 1.8 | (0.2) |
| Macherey - | 17.6 | (16) | 2.1 | (0.4) |
| cells | | | | |
| Macherey - | 22.7 | (24.3) | 1.9 | (0.20) |
| serum/plasma | | | | |

RNA concentrations in whole saliva are in the range of 17.6 to 40.7 μg/mL after extraction with the Norgen or Macherey-Nagel kits. These values show that saliva contains 30 to 40 fold more RNA as compared to serum.

### 3.2. Quality Control Assessment Using the Agilent 2100 Bioanalyzer

The quality and quantity of the RNA was also evaluated by using the Agilent 2100 Bioanalyzer (Agilent Technologies Inc., Santa Clara, Calif.) using two assays:
the RNA 6000 Nano assay for the total RNA (used principally for cells samples)
the Small RNA assay for small RNA (used for all types of samples)

The RNA 6000 Nano assay was used with on the Agilent 2100 bioanalyzer to determine the integrity and the concentration of Total RNA samples extracted from cells with different protocols.

The data analysis software automatically reports the corresponding RNA concentrations for each sample in a range between 5 and 500 ng/μl (qualitative) and 25 and 500 ng/μl (quantitative). Moreover, the performance of Agilent 2100 bioanalyzer was compared to the most commonly used techniques for RNA separation, detection and quantitation. Comparisons between different techniques were based on sensitivity and quantitative accuracy. The advantages of detection sensitivity and accuracy, coupled with a rapid and automated system, indicate that analyses performed by Agilent 2100 bioanalyzer are superior to the leading alternatives.

Small nucleic acids ranging in size from 6 to 150 nucleotides can be analyzed running the Small RNA assay on the Agilent 2100 bioanalyzer. The small RNAs fraction (<150 bp) should also contain microRNAs in their primitive (pri-miRNA), precursor (premiRNA) and mature (miRNA) forms.

The Small RNA assay can:
Visualize miRNA, Small RNA, oligo nucleotides from 6-150 nt for verifying sample integrity
Quantify miRNA in the concentration range of 50-2000 pg/μL relative to an external standard, for verifying sample enrichment and purity
Automate sample quantitation, sizing and purity determination

The quality and integrity of the RNA preparations were assessed using the given parameters:

Nanodrop, Fischer scientific: 1,8<(DO 260/280)<2,2

2100 Bioanalyzer, Agilent:

6000 Nano 1,8<(28S/18S)<2,2 and 7<RIN<10
Small RNA (electrophoregram profile), % microRNA
   3.2.1. Circulating RNAs from Serum and Saliva

Mean values (ng/mL) obtained from 10 samples of small RNA. Samples of saliva from healthy donors were analysed on the small RNA chip. Standard deviation is indicated in brackets.

miRNA in Serum

| Mean values | Small RNA | | | | Ratio miRNA/ | |
|---|---|---|---|---|---|---|
| (n = 10) | (total) | | miRNAs | | Total (%) | |
| Qiagen | 47.70 | (27.70) | 22.00 | (15.50) | 46.10 | (12.10) |
| Ambion | 144.00 | (164.80) | 55.60 | (59.30) | 38.60 | (13.60) |
| Macherey Nagel- | 162.50 | (109.60) | 99.10 | (88.80) | 61.00 | (24.70) |
| Serum/Plasma | | | | | | |
| Norgen | 49.30 | (17.00) | 18.60 | (10.20) | 37.70 | (15.50) |

From sera, total Small RNA concentrations are in the range of 47.70 to 162.50 ng/mL. The samples contain a high percentage of miRNA (37.70 to 61.00%), as measured with the Macherey-Nagel kit.

miRNA in Saliva

| Mean values | Small RNA | | | | Ratio miRNA/ | |
|---|---|---|---|---|---|---|
| (n = 10) | (total) | | miRNAs | | Total (%) | |
| Macherey Nagel- | 7.00 | (7.90) | 4.20 | (5.20) | 59.40 | (13.30) |
| Serum/Plasma | | | | | | |
| Macherey Nagel - | 5.70 | (3.60) | 1.80 | (1.00) | 34.80 | (7.30) |
| Cells | | | | | | |
| Norgen | 6.50 | (4.50) | 2.00 | (1.40) | 30.00 | (5.00) |

In whole saliva, total Small RNA concentrations are in the range of 5.70 to 7.00 ng/mL. The samples contain a high percentage of miRNA (30.00 to 59.40%).

3.2.2. Analysis of Circulating microRNAs in Sera, and Saliva, as Compared to PBMCs

### Examples on Serums

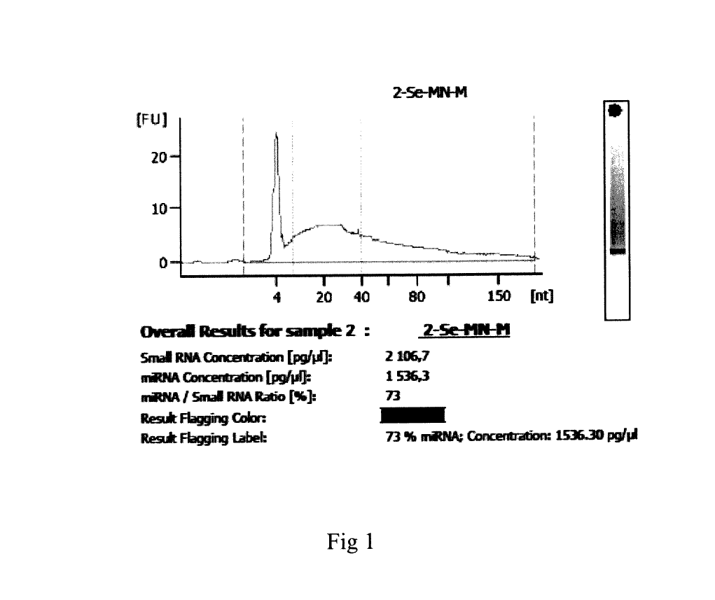

RNAs were extracted from the serum of donor 2 using the Qiagen kit (FIG. 1, high panel) or the Macherey-Nagel kit (FIG. 1, low panel), respectively. Analysis on the Small RNA chip. The results presented FIG. 1 show that the miRNA correspond to 50% (high panel) or 73% (low panel) of total RNA extracted from the serum.

### Examples on Saliva

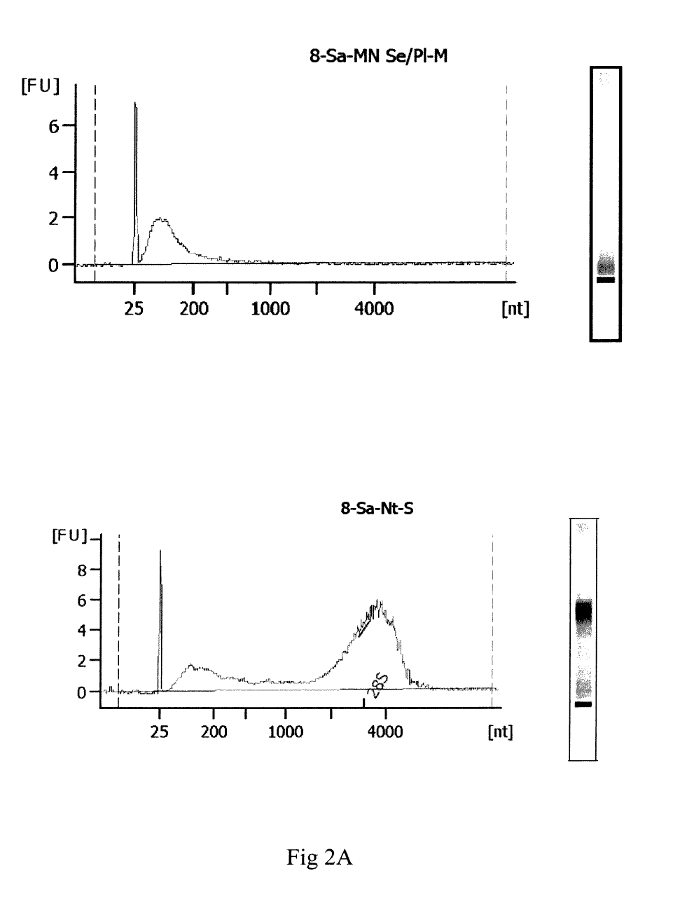

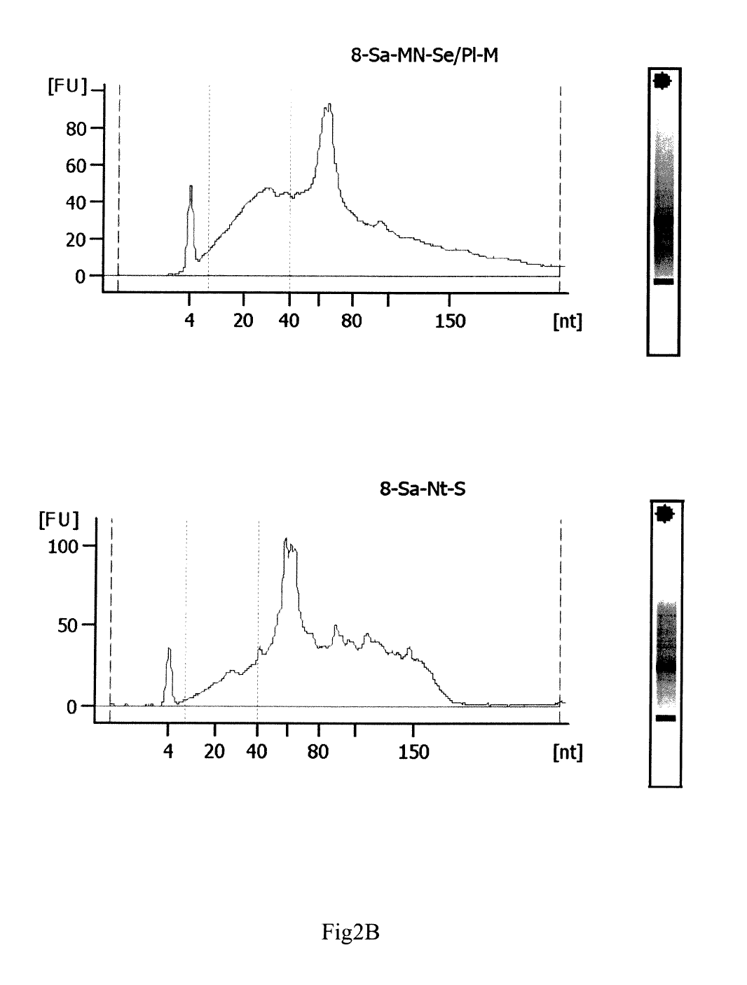

RNAs were extracted from the saliva of a healthy donor (#8) using the Macherey-Nagel (FIG. 2, left panel) or the Norgen kit (FIG. 2, right panel), respectively. Analysis was carried out on Agilent 6000 (FIG. 2A) or Agilent Small RNA (FIG. 2B).

FIG. 2 shows that small RNA can be identified from saliva. The extract obtained with the Norgen kit also shows large RNA. FIG. 2B shows that miRNA correspond to between approx. 60% (left panel) or 33% (right panel) of total RNA extracted from saliva.

### Examples on PBMCs

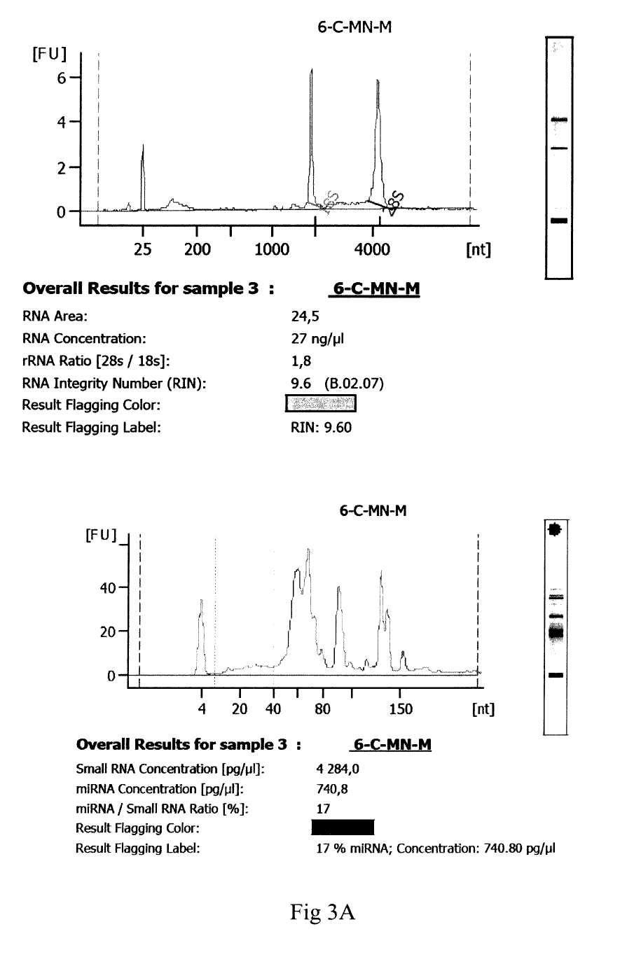

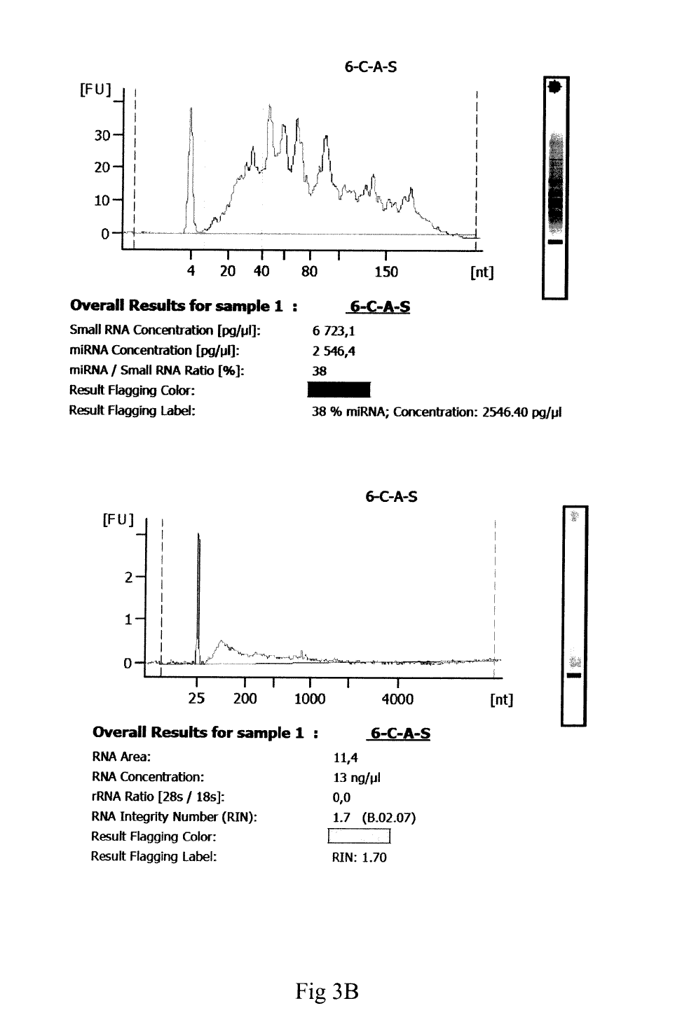

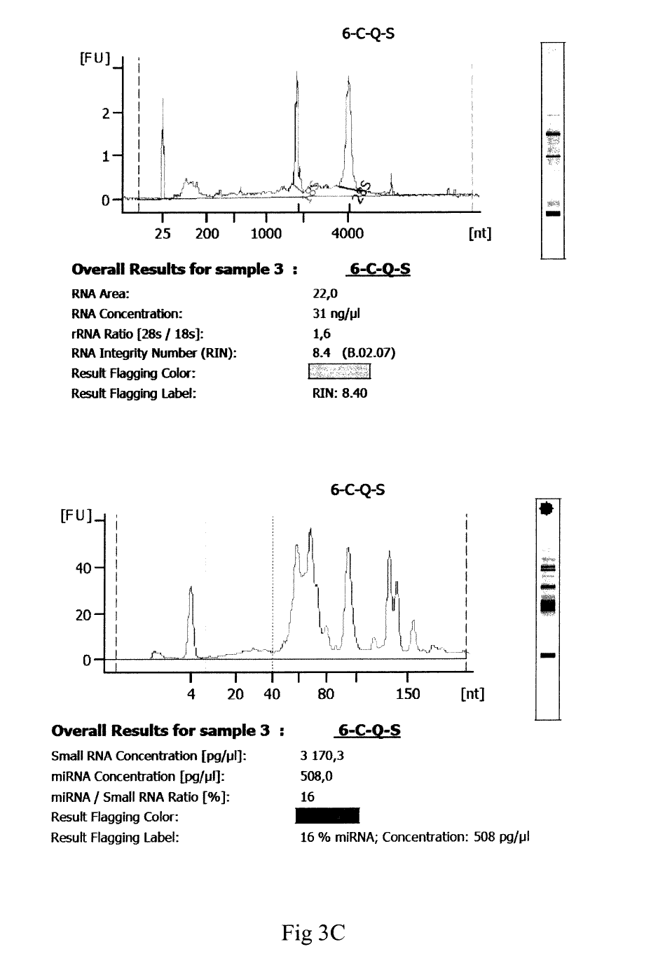

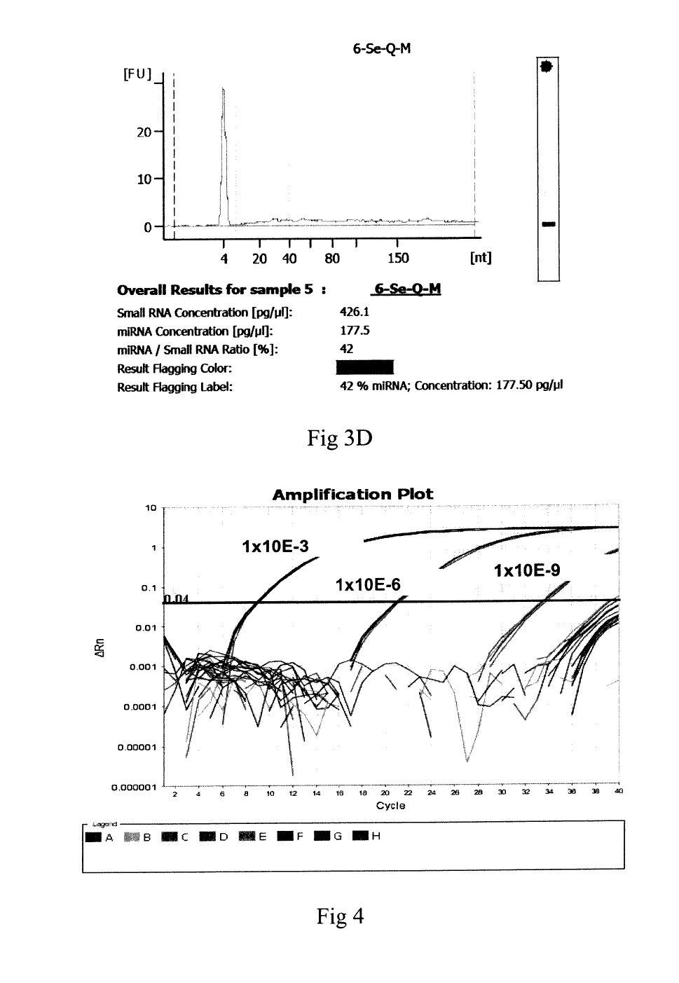

PBMCs were extracted from donors and the microRNAs in circulating cells were determined using different kits. The results are presented in FIG. 3A-D.

### Discussion

The results show circulating microRNAs can be efficiently detected in fluids such as saliva or serum as well as in circulating blood cells. The results further show that circulating microRNAs represent a very substantial portion of total circulating RNAs, of approx. 50% in serum and saliva. These fluids therefore represent advantageous samples for testing microRNAs. Our results also show that microRNAs in serum may be detected with a high level of quality and low contamination by other populations of RNAs. In addition, our results further show that microRNAs are stable and may be tested in fluids even after freezing-thawing the samples.

### 3.3. Quality Control Assessment of Mi-RNA Using RT-qPCR

Various methods of measuring the levels or amounts of miRNAs are available. Any specific method can be used: the level of miRNA is measured during the amplification process or, alternatively, miRNA is amplified prior to measurement. In other methods, the miRNA is not amplified prior to measurement.

Amplification reactions involving suitable nucleic acid polymerization and amplification techniques include reverse transcription (RT), polymerase chain reaction (PCR), real-time PCR (quantitative PCR (q-PCR)), nucleic acid sequence-base amplification (NASBA), and others.

More than one amplification method can be used: reverse transcription followed by real time quantitative PCR (qRT-PCR). A typical PCR reaction includes multiple amplification steps, or cycles that selectively amplify target nucleic acid species: a denaturing step in which a target nucleic acid is denatured; an annealing step in which a set of PCR primers (forward and reverse primers) anneal to complementary DNA strands; and an elongation step in which a thermostable DNA polymerase elongates the primers. By repeating these steps multiple times, a DNA fragment is amplified to produce an amplicon, corresponding to the target DNA sequence. Typical PCR reactions include 20 or more cycles of denaturation, annealing, and elongation. Since mature miRNAs are single-stranded, a reverse transcription reaction that leads in the production of a complementary cDNA sequence is performed prior to PCR reactions. The reverse transcription reactions includes the use of a RNA-based DNA polymerase (reverse transcriptase) and a primer.

Quantitative RT-PCR (qRT-PCR)

Quality control was performed by measuring the expression of miR-638, with individual TaqMan miRNA assay (Applied Bio systems/Life Technologies, Carlsbad, Calif., USA). The method is performed according to the recommendation of the manufacturer; it uses the following reagents: TaqMan MicroRNA Reverse Transcription Kit (ref 4366596), TaqMan® Universal Master Mix II, no UNG 1-Pack (ref 4440043), and TaqMan® MicroRNA Assays (assay id=001582; ref 4440887). The TaqMan® MicroRNA Assay reagent contains specific miRNA-638 primers (agggaucgcgggcggguggcggccu; SEQ ID NO: 179).

The results are presented in FIG. 4. They show that a synthetic miRNA-638 can be detected at very low concentrations of 1×10E-3, 1×10E-6, and 1×10E-9 μg/μL after 9, 22, and 34 cycles, respectively.

Identification of miRNA-638 in Serums of Donors

Total miRNAs isolated from the serum of healthy human donors were subjected to RT-qPCR. Ten ng of total mi-RNA extracted with the preparation kits (Qiagen; Macherey-Nagel) were used in the assay.

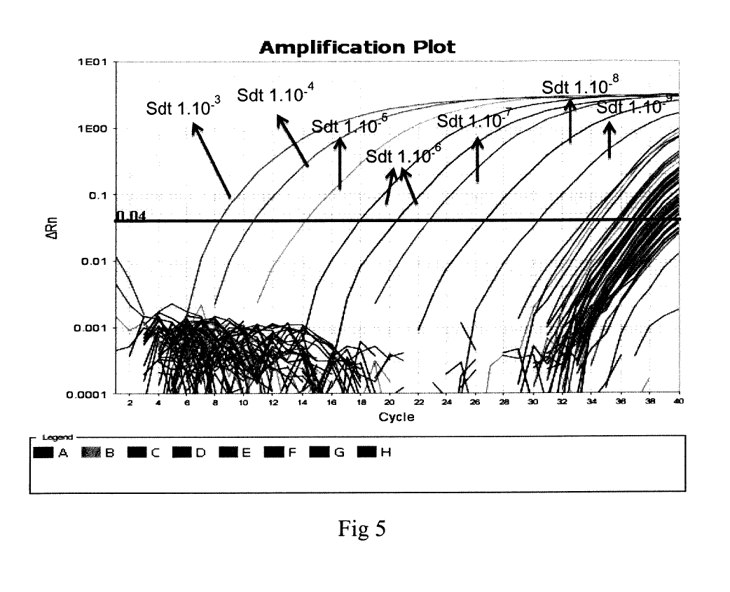

The results are presented in FIG. 5.

They show that microRNA-638 from the serum of patients can be identified (see the pattern on the right side). The concentration is below 1.10⁻⁹ μg/μL.

The exact quantification of miRNA-638 was performed in several samples and is reported in the Table below.

Quantification of miRNA-638 (Femtograms) Obtained from Serum, Saliva, and PBMCs of Healthy Donors (TaqMan, Life-Technologies)

| | | | | |
|---|---|---|---|---|
| | | Macherey | | |
| | Macherey | Nagel | | |
| | Nagel | (serum/ | | |
| Qiagen | (cells) | plasma) | Ambion | Norgen |

PBMC

| Nb of tested | 10 | | 10 | | n.t. | 9 | |
|---|---|---|---|---|---|---|---|
| samples | | | | | | | |
| Nb quantified | 6 | | 7 | | n.t. | 9 | |
| samples | | | | | | | |
| Mean quantity | 12.00 | (7.54) | 10.00 | (5.41) | n.t. | 61.50 | (26.20) |
| miR-638(fg)/ | | | | | | | |
| 1 · 10⁶ | | | | | | | |
| PBMC (SD) | | | | | | | |

SERUM

| Nb of tested | 10 | | NA | 10 | | 10 | |
|---|---|---|---|---|---|---|---|
| samples | | | | | | | |
| Nb quantified | 10 | | NA | 10 | | 3 | |
| samples | | | | | | | |
| Mean quantity | 7.73 | (3.13) | NA | 21.60 | (17.50) | 52.89 | (23.18) |
| miR-638(fg)/ | | | | | | | |
| mL SD) | | | | | | | |

SALIVA

| Nb tested samples | 4 | | 4 | | 4 |
|---|---|---|---|---|---|
| Nb quantified | 4 | | 4 | | 4 |
| samples | | | | | |
| Mean quantity | 98.07 | (26.16) | 558.90 | (255.57) | 66.67 (33.49) |
| miR-638(fg)/ | | | | | |
| mL (SD) | | | | | |

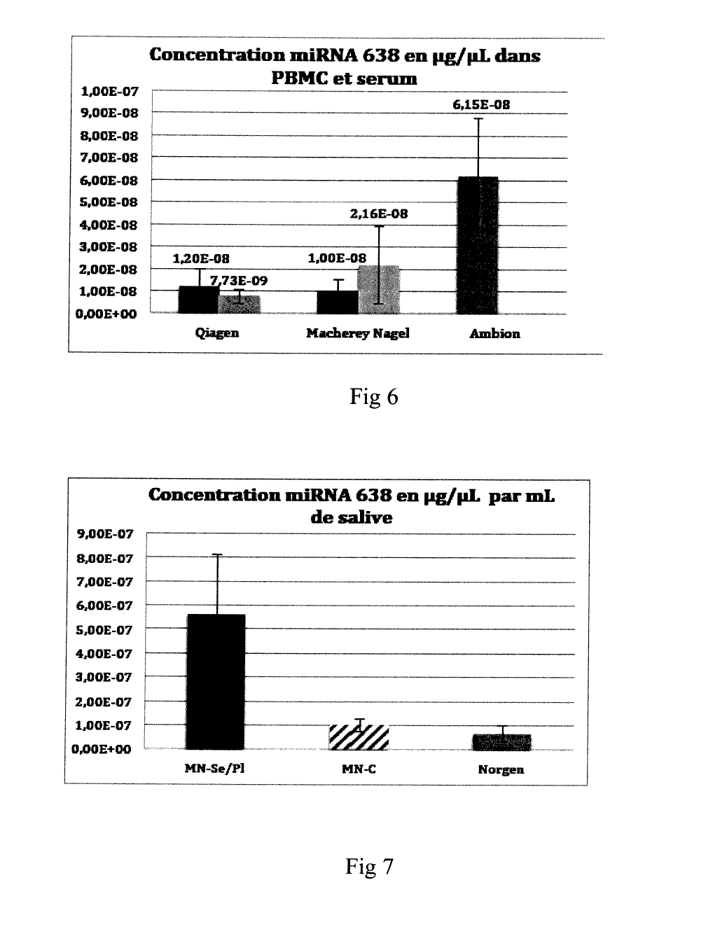

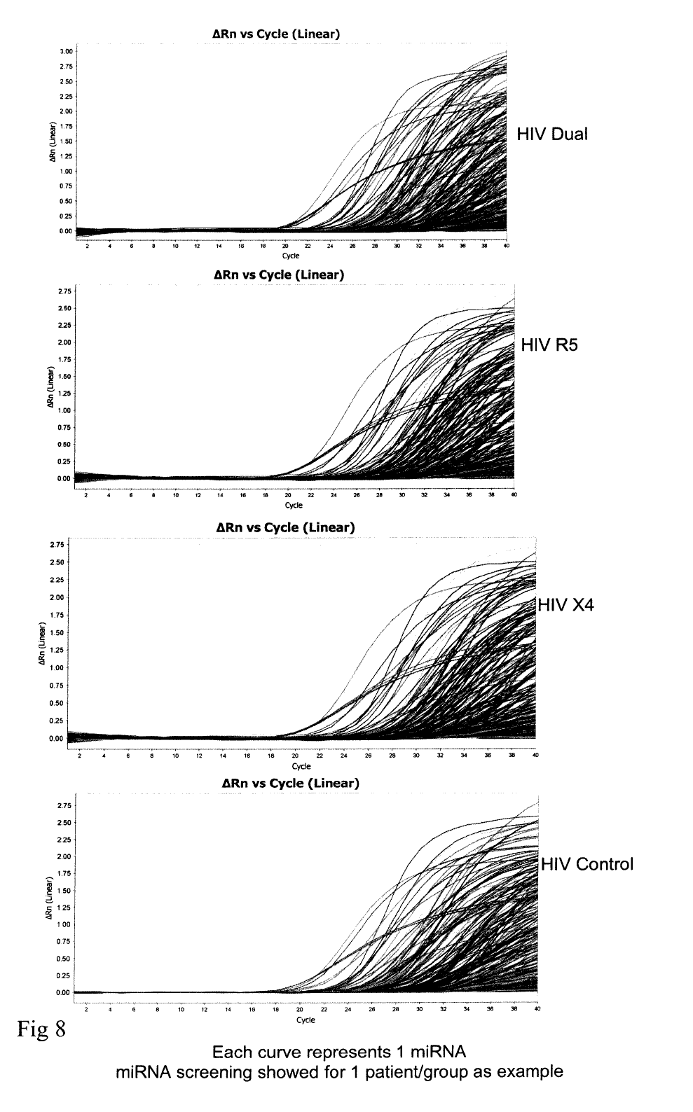

The results are also reported in FIGS. 6 and 7.

The above examples disclose the conditions allowing efficient detection of circulating microRNAs for detecting viral tropism. The results show circulating microRNAs can be efficiently detected in fluids such as saliva or serum. Our results further show that circulating microRNAs represent a very substantial portion of total circulating RNAs, of approx. 50% in serum and saliva, and that microRNAs in serum may be detected with a high level of quality and low contamination by other populations of RNAs. In addition, our results further show that microRNAs are stable and may be tested in fluids even after freezing-thawing the samples. Our results also demonstrate that miRNA-638 can be detected in fluid samples, and exactly quantified. They further show that this specific microRNA represents approx. 0.1% of all circulating RNAs in the serum and may be reliably detected and used to analyze virus tropism in patients infected with a virus.

4. miRNA Screening by RT-qPCR Using TaqMan Array Cards

Profiling was performed using TaqMan Array Human MicroRNA panels A and B (Applied Biosystems, CA). The TLDA cards detect 384 features on each card. In total 754 human miRNAs were tested. Reverse transcription and qPCR were performed with the manufacturer's reagents following instructions (Applied Biosystems, CA). Briefly, 3 μl of PPP sample or 150 ng of RNA from PBMC are used for Megaplex reverse transcription (RT) reaction. Real time quantitative PCR was performed with ViiA7 real-time PCR machine, and data were collected with the manufacturer's ViiA™ Software. Gene Expression Suite software (Applied Biosystems, CA) was used to process the array data. Thresholds at 0.1 were checked individually and corrected as necessary.

Screenings were performed on four groups: patients infected with R5, or X4 or Dual HIV strains (tropism determined by Geno2Pheno algorithm) and a control group of healthy donors (10 individuals minimum/group).

Significant relative quantifications of miRNA between the different groups were analyzed using different normalization and statistical methods: Gene Expression Suite software (Applied Bio systems, CA) and Mann-Whitney test. Significant fold changes were determined by a p value <0.05 and a fold change inferior or superior to 1.

### Patients Description

| | HIV R5 | | HIV X4 | | HIV Dual | | Control | Tot |
|---|---|---|---|---|---|---|---|---|
| n tot | 12 | | 11 | | 10 | | 10 | 43 |
| n Men | 10 | | 9 | | 7 | | 7 | 33 |
| mean age (SD) | 52.2 | (17.2) | 45.3 | (8.7) | 45.7 | (8.2) | 49.2 (10.7) | 48.7 (12.2) |
| mean CD4 (/mm3) | 607 | (558) | 513 | (172) | 636 | (386) | | |
| n detectable VL | 7 | | 7 | | 7 | | | |
| mean VL (cp/mL) | 21 202 | (53 454) | 255 | (443) | 123 941 | (325 385) | | |

VL = viral load

Identification of Further Circulating microRNAs Correlated to Virus Co-Receptor Usage

Following the methods disclosed in section 1, a list of preferred, significantly modulated circulating microRNAs were identified, which can serve to detect or characterize viral infection in human subjects. The list is provided in Table I below.

| | | SEQ |
|---|---|---|
| microRNA | Sequence (in DNA) | ID NO: |
| hsa-let-7f-2# | CTATACAGTCTACTGTCTTTCC | 1 |
| hsa-miR-106a | AAAAGTGCTTACAGTGCAGGTAG | 2 |
| hsa-miR-1208 | TCACTGTTCAGACAGGCGGA | 3 |
| hsa-miR-1233 | TGAGCCCTGTCCTCCCGCAG | 4 |
| hsa-miR-1243 | AACTGGATCAATTATAGGAGTG | 5 |
| hsa-miR-1262 | ATGGGTGAATTTGTAGAAGGAT | 6 |
| hsa-miR-1267 | CCTGTTGAAGTGTAATCCCCA | 7 |
| hsa-miR-1276 | TAAAGAGCCCTGTGGAGACA | 8 |
| hsa-miR-1285 | TCTGGGCAACAAAGTGAGACCT | 9 |
| hsa-miR-1290 | TGGATTTTTGGATCAGGGA | 10 |
| hsa-miR-1298 | TTCATTCGGCTGTCCAGATGTA | 11 |
| hsa-miR-1305 | TTTTCAACTCTAATGGGAGAGA | 12 |
| hsa-miR-140-3p | TACCACAGGGTAGAACCACGG | 13 |
| hsa-miR-142-3p | TGTAGTGTTTCCTACTTTATGGA | 14 |
| hsa-miR-144# | GGATATCATCATATACTGTAAG | 15 |
| hsa-miR-146a | TGAGAACTGAATTCCATGGGTT | 16 |
| hsa-miR-150 | TCTCCCAACCCTTGTACCAGTG | 17 |
| hsa-miR-17 | CAAAGTGCTTACAGTGCAGGTAG | 18 |
| hsa-miR-186 | CAAAGAATTCTCCTTTTGGGCT | 19 |
| hsa-miR-20a | TAAAGTGCTTATAGTGCAGGTAG | 20 |
| hsa-miR-210 | CTGTGCGTGTGACAGCGGCTGA | 21 |
| hsa-miR-222 | AGCTACATCTGGCTACTGGGT | 22 |
| hsa-miR-223 | TGTCAGTTTGTCAAATACCCCA | 23 |
| hsa-miR-24 | TGGCTCAGTTCAGCAGGAACAG | 24 |
| hsa-miR-24-2# | TGCCTACTGAGCTGAAACACAG | 25 |
| hsa-miR-26b | TTCAAGTAATTCAGGATAGGT | 26 |
| hsa-miR-30a-3p | CTTTCAGTCGGATGTTTGCAGC | 27 |
| hsa-miR-30a-5p | TGTAAACATCCTCGACTGGAAG | 28 |
| hsa-miR-30c | TGTAAACATCCTACACTCTCAGC | 29 |
| hsa-miR-320 | AAAAGCTGGGTTGAGAGGGCGA | 30 |
| hsa-miR-323-3p | CACATTACACGGTCGACCTCT | 31 |
| hsa-miR-338-5P | AACAATATCCTGGTGCTGAGTG | 32 |
| hsa-miR-33a | GTGCATTGTAGTTGCATTGCA | 33 |
| hsa-miR-33b | GTGCATTGCTGTTGCATTGC | 34 |
| hsa-miR-340 | TTATAAAGCAATGAGACTGATT | 35 |
| hsa-miR-342-3p | TCTCACACAGAAATCGCACCCGT | 36 |
| hsa-miR-378 | ACTGGACTTGGAGTCAGAAGG | 37 |
| hsa-miR-424 | CAGCAGCAATTCATGTTTTGAA | 38 |
| hsa-miR-483-5p | AAGACGGGAGGAAAGAAGGGAG | 39 |
| hsa-miR-484 | TCAGGCTCAGTCCCCTCCCGAT | 40 |
| hsa-miR-485-3p | GTCATACACGGCTCTCCTCTCT | 41 |
| hsa-miR-486 | TCCTGTACTGAGCTGCCCCGAG | 42 |
| hsa-miR-486-3p | CGGGGCAGCTCAGTACAGGAT | 43 |
| hsa-miR-502 | ATCCTTGCTATCTGGGTGCTA | 44 |
| hsa-miR-550 | AGTGCCTGAGGGAGTAAGAGCCC | 45 |
| hsa-miR-557 | GTTTGCACGGGTGGGCCTTGTCT | 46 |
| hsa-miR-572 | GTCCGCTCGGCGGTGGCCCA | 47 |
| hsa-miR-575 | GAGCCAGTTGGACAGGAGC | 48 |
| hsa-miR-581 | TCTTGTGTTCTCTAGATCAGT | 49 |
| hsa-miR-582-3p | TAACTGGTTGAACAACTGAACC | 50 |
| hsa-miR-584 | TTATGGTTTGCCTGGGACTGAG | 51 |
| hsa-miR-586 | TATGCATTGTATTTTTAGGTCC | 52 |
| hsa-miR-596 | AAGCCTGCCCGGCTCCTCGGG | 53 |
| hsa-miR-597 | TGTGTCACTCGATGACCACTGT | 54 |
| hsa-miR-625# | GACTATAGAACTTTCCCCCTCA | 55 |
| hsa-miR-630 | AGTATTCTGTACCAGGGAAGGT | 56 |
| hsa-miR-638 | AGGGATCGCGGGCGGGTGGCGGCCT | 57 |
| hsa-miR-645 | TCTAGGCTGGTACTGCTGA | 58 |
| hsa-miR-648 | AAGTGTGCAGGGCACTGGT | 59 |
| hsa-miR-656 | AATATTATACAGTCAACCTCT | 60 |
| hsa-miR-657 | GGCAGGTTCTCACCCTCTCTAGG | 61 |
| hsa-miR-659 | CTTGGTTCAGGGAGGGTCCCCA | 62 |
| hsa-miR-661 | TGCCTGGGTCTCTGGCCTGCGCGT | 63 |
| hsa-miR-720 | TCTCGCTGGGGCCTCCA | 64 |
| hsa-miR-770-5p | TCCAGTACCACGTGTCAGGGCCA | 65 |
| hsa-miR-875-5p | TATACCTCAGTTTTATCAGGTG | 66 |
| hsa-miR-892b | CACTGGCTCCTTTCTGGGTAGA | 67 |
| hsa-miR-99b# | CAAGCTCGTGTCTGTGGGTCCG | 68 |
| mmu-miR-451 | AAACCGTTACCATTACTGAGTT | 69 |
| mmu-miR-93 | CAAAGTGCTGTTCGTGCAGGTAG | 70 |

Remarks:
mmu-miR-93 corresponds to human hsa-miR-93-5p
mmu-miR-451 corresponds to human hsa-miR-451a

Preferred circulating microRNAs for use as markers in the present invention are listed below: hsa-miR-106a; hsa-miR-1208; hsa-miR-1233; hsa-miR-1267; hsa-miR-1290; hsa-miR-146a; hsa-miR-150; hsa-miR-17; hsa-miR-186; hsa-miR-20a; hsa-miR-222; hsa-miR-223; hsa-miR-24; hsa-miR-26b; hsa-miR-30a-5p; hsa-miR-30c; hsa-miR-323-3p; hsa-miR-338-5P; hsa-miR-33b; hsa-miR-342-3p; hsa-miR-378; hsa-miR-424; hsa-miR-483-5p; hsa-miR-484; hsa-miR-486; hsa-miR-486-3p; hsa-miR-572; hsa-miR-596; hsa-miR-625#; hsa-miR-638; hsa-miR-659; hsa-miR-661; hsa-miR-99b#; or mmu-miR-93.

The modulation of these microRNAs in infected subjects is detailed in Tables II and III below:

| VS Control | | | VS R5 | | VS X4 |
|---|---|---|---|---|---|
| R5 | X4 | D | X4 | D | D |
| | | hsa-miR-323-3p- | | hsa-miR-323-3p- | hsa-miR-323-3p- |
| | | 002227 | | 002227 | 002227 |
| hsa-miR-146a- | hsa-miR-146a- | hsa-miR-146a- | | | |
| 000468 | 000468 | 000468 | | | |
| | | hsa-miR-186- | | hsa-miR-186- | hsa-miR-186- |
| | | 002285 | | 002285 | 002285 |
| hsa-miR-661- | hsa-miR-661- | | hsa-miR-661- | hsa-miR-661- | |
| 001606 | 001606 | | 001606 | 001606 | |
| hsa-miR-483-5p- | hsa-miR-483-5p- | hsa-miR-483-5p- | | | |
| 002338 | 002338 | 002338 | | | |
| | | hsa-miR-99b#- | | hsa-miR-99b#- | hsa-miR-99b#- |
| | | 002196 | | 002196 | 002196 |
| | | hsa-miR-1233- | | hsa-miR-1233- | |
| | | 002768 | | 002768 | |
| hsa-miR-30a-5p- | | | hsa-miR-30a-5p- | hsa-miR-30a-5p- | |
| 000417 | | | 000417 | 000417 | |
| hsa-miR-486- | | | hsa-miR-486- | hsa-miR-486- | |
| 001278 | | | 001278 | 001278 | |
| hsa-miR-222- | hsa-miR-222- | hsa-miR-222- | | | |
| 002276 | 002276 | 002276 | | | |
| | | hsa-miR-24- | | hsa-miR-24- | |
| | | 000402 | | 000402 | |
| | | hsa-miR-1290- | | hsa-miR-1290- | hsa-miR-1290- |
| | | 002863 | | 002863 | 002863 |
| | hsa-miR-659- | hsa-miR-659- | hsa-miR-659- | hsa-miR-659- | hsa-miR-659- |
| | 001514 | 001514 | 001514 | 001514 | 001514 |
| hsa-miR-26b- | | hsa-miR-26b- | | | |
| 000407 | | 000407 | | | |
| hsa-miR-17- | | hsa-miR-17- | | | |
| 002308 | | 002308 | | | |
| hsa-miR-106a- | | hsa-miR-106a- | | | |
| 002169 | | 002169 | | | |
| | | | hsa-miR-484- | hsa-miR-484- | |
| | | | 001821 | 001821 | |
| hsa-miR-638- | | | hsa-miR-638- | hsa-miR-638- | |
| 001582 | | | 001582 | 001582 | |
| | | | hsa-miR-625#- | hsa-miR-625#- | |
| | | | 002432 | 002432 | |
| | | hsa-miR-596- | hsa-miR-596- | hsa-miR-596- | |
| | | 001550 | 001550 | 001550 | |
| | hsa-miR-150- | — | hsa-miR-150- | hsa-miR-150- | |
| | 000473 | | 000473 | 000473 | |
| | | — | | | hsa-miR-223- |
| | | | | | 002295 |
| mmu-miR-93- | | mmu-miR-93- | | | |
| 001090 | | 001090 | | | |
| hsa-miR-20a- | | hsa-miR-20a- | | | |
| 000580 | | 000580 | | | |
| | | hsa-miR-1267- | | hsa-miR-1267- | hsa-miR-1267- |
| | | 002885 | | 002885 | 002885 |
| | | | | hsa-miR-378- | hsa-miR-378- |
| | | | | 002243 | 002243 |
| | | hsa-miR-338-5P- | | hsa-miR-338-5P- | hsa-miR-338-5P- |
| | | 002658 | | 002658 | 002658 |
| | | | hsa-miR-486-3p- | — | |
| | | | 002093 | | |
| | | hsa-miR-33b- | | hsa-miR-33b- | hsa-miR-33b- |
| | | 002085 | | 002085 | 002085 |
| | | hsa-miR-424- | | hsa-miR-424- | hsa-miR-424- |
| | | 000604 | | 000604 | 000604 |
| hsa-miR-572- | | | hsa-miR-572- | hsa-miR-572- | |
| 001614 | | | 001614 | 001614 | |
| | hsa-miR-30c- | | hsa-miR-30c- | | |
| | 000419 | | 000419 | | |
| | | | hsa-miR-342-3p- | | |
| | | | 002260 | | |
| hsa-miR-1208- | | hsa-miR-1208- | | | |
| 002880 | | 002880 | | | |

lower-expression
over-expression

| | R5 | | X4 | | Dual | | Control | |
|---|---|---|---|---|---|---|---|---|
| | mean Ct | sd | mean Ct | sd | mean Ct | sd | mean Ct | sd |
| hsa-miR-106a | 29.100 | 1.2412 | 28.171 | 1.6506 | 28.760 | 0.8564 | 27.816 | 0.9530 |
| hsa-miR-1208 | 32.364 | 1.4294 | 34.433 | 1.3941 | 33.147 | 1.2537 | 34.014 | 1.1264 |
| hsa-miR-1233 | 28.501 | 1.4745 | 29.797 | 1.3878 | 31.242 | 1.9217 | 29.183 | 0.9427 |
| hsa-miR-1267 | 32.515 | 2.7468 | 31.553 | 1.0688 | 28.677 | 0.8624 | 31.998 | 1.7467 |
| hsa-miR-1290 | 27.704 | 1.9560 | 28.054 | 1.0137 | 26.744 | 0.8942 | 28.410 | 0.9770 |
| hsa-miR-146a | 30.031 | 1.2115 | 29.768 | 0.8134 | 29.458 | 1.0494 | 31.079 | 1.5559 |
| hsa-miR-150 | 30.589 | 0.8544 | 29.875 | 0.5837 | 30.170 | 1.3940 | 30.802 | 0.9923 |
| hsa-miR-17 | 29.009 | 1.1793 | 28.201 | 1.4990 | 28.798 | 0.9602 | 27.916 | 0.9705 |
| hsa-miR-186 | 31.064 | 1.2073 | 31.242 | 1.3147 | 30.181 | 0.9247 | 31.916 | 1.6564 |
| hsa-miR-20a | 30.608 | 1.3795 | 29.911 | 1.7201 | 31.182 | 1.0620 | 29.560 | 1.4815 |
| hsa-miR-222 | 30.201 | 1.7882 | 30.093 | 0.9880 | 29.903 | 0.9299 | 31.239 | 0.9482 |
| hsa-miR-223 | 25.596 | 1.6417 | 26.129 | 1.2483 | 24.978 | 1.0138 | 25.936 | 1.0972 |
| hsa-miR-24 | 29.908 | 1.2276 | 29.557 | 1.2509 | 29.242 | 0.8181 | 30.475 | 1.1187 |
| hsa-miR-26b | 32.125 | 1.7450 | 31.251 | 1.2768 | 32.606 | 2.1378 | 30.553 | 1.3997 |
| hsa-miR-30a-5p | 28.810 | 0.8965 | 30.150 | 1.4180 | 30.507 | 0.8140 | 29.917 | 0.5486 |
| hsa-miR-30c | 33.333 | 1.5975 | 32.687 | 1.0579 | 33.538 | 2.2225 | 33.101 | 1.1582 |
| hsa-miR-323-3p | 29.620 | 0.5948 | 29.351 | 0.4291 | 33.162 | 1.5210 | 29.342 | 0.2168 |
| hsa-miR-338-5P | 30.826 | 0.3078 | 30.909 | 0.4396 | 32.021 | 0.3355 | 30.520 | 0.2547 |
| hsa-miR-33b | | | | | 31.801 | 1.8571 | | |
| hsa-miR-342-3p | 32.235 | 0.9923 | 31.920 | 0.6152 | 33.182 | 1.4832 | 32.335 | 0.9257 |
| hsa-miR-378 | 30.027 | 0.8638 | 30.669 | 1.4439 | 31.670 | 0.7533 | 30.878 | 1.0004 |
| hsa-miR-424 | | | | | 29.733 | 1.8699 | | |
| hsa-miR-483-5p | 31.355 | 1.4732 | 32.229 | 1.7098 | 31.939 | 1.2839 | 33.051 | 1.5235 |
| hsa-miR-484 | 29.013 | 1.0220 | 28.013 | 1.2049 | 28.358 | 1.3002 | 28.542 | 1.2042 |
| hsa-miR-486 | 25.813 | 0.9611 | 24.734 | 1.4779 | 24.949 | 1.1283 | 24.903 | 0.6815 |
| hsa-miR-486-3p | 31.770 | 1.9956 | 30.383 | 1.2127 | 31.564 | 2.3186 | 31.262 | 2.0243 |
| hsa-miR-572 | 32.087 | 2.1697 | 34.423 | 2.5084 | 34.307 | | 35.101 | 2.4164 |
| hsa-miR-596 | 27.417 | 0.9252 | 29.057 | 0.6805 | 30.795 | 0.6188 | 28.144 | 0.6646 |
| hsa-miR-625# | 30.351 | 2.4156 | 33.424 | 2.1801 | 34.379 | 2.8147 | 32.646 | 2.1614 |
| hsa-miR-638 | 30.394 | 0.8443 | 32.521 | 1.1805 | 32.223 | 0.6049 | 31.872 | 0.9357 |
| hsa-miR-659 | 29.929 | 1.2114 | 32.762 | 0.8808 | 36.485 | 1.6162 | 30.444 | 1.0498 |
| hsa-miR-661 | 25.595 | 2.6260 | 28.751 | 2.2822 | 28.035 | 2.1298 | 27.407 | 2.4428 |
| hsa-miR-99b# | 31.042 | 0.6474 | 31.619 | 0.5424 | 34.780 | 1.2875 | 31.126 | 0.2976 |
| mmu-miR-93 | 31.130 | 1.9443 | 30.275 | 2.1670 | 31.087 | 1.1835 | 29.570 | 0.7698 |

Ct values obtained by RTqPCR on array cards.

Identification of Further PBMC microRNAs Correlated to Virus Co-Receptor Usage

Following the methods disclosed in section 1, a list of preferred, significantly modulated, microRNAs in PBMCs were identified, which can serve to detect or characterize viral infection in human subjects. The list is provided in Table IV below.

| microRNA | Sequence (in DNA) | SEQ ID NO: |
|---|---|---|
| hsa-let-7a | TGAGGTAGTAGGTTGTATAGTT | 71 |
| hsa-let-7d | AGAGGTAGTAGGTTGCATAGTT | 72 |
| hsa-let-7e | TGAGGTAGGAGGTTGTATAGTT | 73 |
| hsa-let-7f | TGAGGTAGTAGATTGTATAGTT | 74 |
| hsa-let-7g | TGAGGTAGTAGTTTGTACAGTT | 75 |
| hsa-miR-103 | AGCAGCATTGTACAGGGCTATGA | 76 |
| hsa-miR-106a | AAAAGTGCTTACAGTGCAGGTAG | 77 |
| hsa-miR-106b | TAAAGTGCTGACAGTGCAGAT | 78 |
| hsa-miR-124-3p (mmu-miR-124a) | TAAGGCACGCGGTGAATGCC | 79 |
| hsa-miR-1244 | AAGTAGTTGGTTTGTATGAGATGGTT | 80 |
| hsa-miR-126 | TCGTACCGTGAGTAATAATGCG | 81 |
| hsa-miR-126# | CATTATTACTTTTGGTACGCG | 82 |
| hsa-miR-1260 | ATCCCACCTCTGCCACCA | 83 |
| hsa-miR-1274A | GTCCCTGTTCAGGCGCCA | 84 |
| hsa-miR-1276 | TAAAGAGCCCTGTGGAGACA | 85 |
| hsa-miR-130a | CAGTGCAATGTTAAAAGGGCAT | 86 |
| hsa-miR-130b | CAGTGCAATGATGAAAGGGCAT | 87 |
| hsa-miR-133a | TTTGGTCCCCTTCAACCAGCTG | 88 |
| hsa-miR-134 (mmu-miR-134) | TGTGACTGGTTGACCAGAGGGG | 89 |
| hsa-miR-139-5p | TCTACAGTGCACGTGTCTCCAG | 90 |
| hsa-miR-140-5p (mmu-miR-140) | CAGTGGTTTTACCCTATGGTAG | 91 |
| hsa-miR-142-3p | TGTAGTGTTTCCTACTTTATGGA | 92 |
| hsa-miR-142-5p | CATAAAGTAGAAAGCACTACT | 93 |
| hsa-miR-146a | TGAGAACTGAATTCCATGGGTT | 94 |
| hsa-miR-146b | TGAGAACTGAATTCCATAGGCT | 95 |
| hsa-miR-148a | TCAGTGCACTACAGAACTTTGT | 96 |
| hsa-miR-148b | TCAGTGCATCACAGAACTTTGT | 97 |
| hsa-miR-149# | AGGGAGGGACGGGGGCTGTGC | 98 |
| hsa-miR-150 | TCTCCCAACCCTTGTACCAGTG | 99 |
| hsa-miR-151-5P | TCGAGGAGCTCACAGTCTAGT | 100 |
| hsa-miR-15b | TAGCAGCACATCATGGTTTACA | 101 |
| hsa-miR-16 | TAGCAGCACGTAAATATTGGCG | 102 |
| hsa-miR-17 | CAAAGTGCTTACAGTGCAGGTAG | 103 |
| hsa-miR-1825 | TCCAGTGCCCTCCTCTCC | 104 |
| hsa-miR-185 | TGGAGAGAAAGGCAGTTCCTGA | 105 |
| hsa-miR-186 | CAAAGAATTCTCCTTTTGGGCT | 106 |
| hsa-miR-18b | TAAGGTGCATCTAGTGCAGTTAG | 107 |
| hsa-miR-191 | CAACGGAATCCCAAAAGCAGCTG | 108 |
| hsa-miR-192 | CTGACCTATGAATTGACAGCC | 109 |
| hsa-miR-195 | TAGCAGCACAGAAATATTGGC | 110 |
| hsa-miR-196b | TAGGTAGTTTCCTGTTGTTGGG | 111 |
| hsa-miR-199a-3p | ACAGTAGTCTGCACATTGGTTA | 112 |
| hsa-miR-19a | TGTGCAAATCTATGCAAAACTGA | 113 |
| hsa-miR-19b | TGTGCAAATCCATGCAAAACTGA | 114 |
| hsa-miR-200b | TAATACTGCCTGGTAATGATGA | 115 |
| hsa-miR-20a | TAAAGTGCTTATAGTGCAGGTAG | 116 |
| hsa-miR-20b | CAAAGTGCTCATAGTGCAGGTAG | 117 |
| hsa-miR-21 | TAGCTTATCAGACTGATGTTGA | 118 |
| hsa-miR-221 | AGCTACATTGTCTGCTGGGTTTC | 119 |
| hsa-miR-223 | TGTCAGTTTGTCAAATACCCCA | 120 |
| hsa-miR-223# | CGTGTATTTGACAAGCTGAGTT | 121 |
| hsa-miR-24-2# | TGCCTACTGAGCTGAAACACAG | 122 |
| hsa-miR-25 | CATTGCACTTGTCTCGGTCTGA | 123 |
| hsa-miR-26a | TTCAAGTAATCCAGGATAGGCT | 124 |
| hsa-miR-26b | TTCAAGTAATTCAGGATAGGT | 125 |
| hsa-miR-27a | TTCACAGTGGCTAAGTTCCGC | 126 |
| hsa-miR-27a# | AGGGCTTAGCTGCTTGTGAGCA | 127 |
| hsa-miR-28 | AAGGAGCTCACAGTCTATTGAG | 128 |
| hsa-miR-299-3p | TATGTGGGATGGTAAACCGCTT | 129 |
| hsa-miR-29a | TAGCACCATCTGAAATCGGTTA | 130 |
| hsa-miR-29c | TAGCACCATTTGAAATCGGTTA | 131 |
| hsa-miR-301 | CAGTGCAATAGTATTGTCAAAGC | 132 |
| hsa-miR-301b | CAGTGCAATGATATTGTCAAAGC | 133 |
| hsa-miR-30b | TGTAAACATCCTACACTCAGCT | 134 |
| hsa-miR-30c | TGTAAACATCCTACACTCTCAGC | 135 |
| hsa-miR-31 | AGGCAAGATGCTGGCATAGCT | 136 |
| hsa-miR-324-3p | ACTGCCCCAGGTGCTGCTGG | 137 |
| hsa-miR-328 | CTGGCCCTCTCTGCCCTTCCGT | 138 |
| hsa-miR-335 | TCAAGAGCAATAACGAAAAATGT | 139 |
| hsa-miR-340 | TTATAAAGCAATGAGACTGATT | 140 |
| hsa-miR-340# | TCCGTCTCAGTTACTTTATAGC | 141 |
| hsa-miR-342-3p | TCTCACACAGAAATCGCACCCGT | 142 |
| hsa-miR-365 | TAATGCCCCTAAAAATCCTTAT | 143 |
| hsa-miR-374 | TTATAATACAACCTGATAAGTG | 144 |
| hsa-miR-374b-5p (mmu-miR-374-5p) | ATATAATACAACCTGCTAAGTG | 145 |
| hsa-miR-376c | AACATAGAGGAAATTCCACGT | 146 |
| hsa-miR-380-5p | TGGTTGACCATAGAACATGCGC | 147 |
| hsa-miR-410 | AATATAACACAGATGGCCTGT | 148 |
| hsa-miR-422a | ACTGGACTTAGGGTCAGAAGGC | 149 |
| hsa-miR-425-5p | AATGACACGATCACTCCCGTTGA | 150 |
| hsa-miR-432 | TCTTGGAGTAGGTCATTGGGTGG | 151 |
| hsa-miR-451a (mmu-miR-451) | AAACCGTTACCATTACTGAGTT | 152 |
| hsa-miR-454 | TAGTGCAATATTGCTTATAGGGT | 153 |
| hsa-miR-486 | TCCTGTACTGAGCTGCCCCGAG | 154 |
| hsa-miR-494 | TGAAACATACACGGGAAACCTC | 155 |
| hsa-miR-495 (mmu-miR-495) | AAACAAACATGGTGCACTTCTT | 156 |
| hsa-miR-516-3p | TGCTTCCTTTCAGAGGGT | 157 |
| hsa-miR-522 | AAAATGGTTCCCTTTAGAGTGT | 158 |
| hsa-miR-532 | CATGCCTTGAGTGTAGGACCGT | 159 |
| hsa-miR-532-3p | CCTCCCACACCCAAGGCTTGCA | 160 |
| hsa-miR-543 | AAACATTCGCGGTGCACTTCTT | 161 |
| hsa-miR-571 | TGAGTTGGCCATCTGAGTGAG | 162 |
| hsa-miR-574-3p | CACGCTCATGCACACACCCACA | 163 |
| hsa-miR-590-3P | TAATTTTATGTATAAGCTAGT | 164 |
| hsa-miR-590-5p | GAGCTTATTCATAAAAGTGCAG | 165 |
| hsa-miR-628-5p | ATGCTGACATATTTACTAGAGG | 166 |
| hsa-miR-638 | AGGGATCGCGGGCGGGTGGCGGCCT | 167 |
| hsa-miR-642 | GTCCCTCTCCAAATGTGTCTTG | 168 |
| hsa-miR-650 | AGGAGGCAGCGCTCTCAGGAC | 169 |
| hsa-miR-651 | TTTAGGATAAGCTTGACTTTTG | 170 |
| hsa-miR-652 | AATGGCGCCACTAGGGTTGTG | 171 |
| hsa-miR-660 | TACCCATTGCATATCGGAGTTG | 172 |
| hsa-miR-7-1-3p (rno-miR-7#) | CAACAAATCACAGTCTGCCATA | 173 |
| hsa-miR-744# | CTGTTGCCACTAACCTCAACCT | 174 |
| hsa-miR-765 | TGGAGGAGAAGGAAGGTGATG | 175 |
| hsa-miR-875-5p | TATACCTCAGTTTTATCAGGTG | 176 |
| hsa-miR-93-5p (mmu-miR-93) | CAAAGTGCTGTTCGTGCAGGTAG | 177 |
| hsa-miR-942 | TCTTCTCTGTTTTGGCCATGTG | 178 |

Preferred PBMCs microRNAs for use as markers in the present invention are listed below: hsa-let-7a; hsa-let-7e; hsa-miR-124-3p (mmu-miR-124a); hsa-miR-126; hsa-miR-126#; hsa-miR-130a; hsa-miR-146b; hsa-miR-150; hsa-miR-17; hsa-miR-186; hsa-miR-191; hsa-miR-199a-3p; hsa-miR-19b; hsa-miR-20a; hsa-miR-20b; hsa-miR-21; hsa-miR-221; hsa-miR-26b; hsa-miR-30b; hsa-miR-30c; hsa-miR-31; hsa-miR-374; hsa-miR-376c; hsa-miR-432; hsa-miR-451a (mmu-miR-451); hsa-miR-454; hsa-miR-486; hsa-miR-494; hsa-miR-495 (mmu-miR-495); hsa-miR-516-3p; hsa-miR-522; hsa-miR-574-3p; hsa-miR-7-1-3p (rno-miR-7#); or hsa-miR-875-5p.

The modulation of these microRNAs in infected subjects is detailed in Tables V and VI below:

TABLE V

| VS Control | | | VS R5 | | VS X4 |
|---|---|---|---|---|---|
| R5 | X4 | D | X4 | D | D |
| mmu-miR-124a- | mmu-miR-124a- | mmu-miR-124a- | | | |
| 001182 | 001182 | 001182 | | | |
| hsa-miR-494- | | hsa-miR-494- | hsa-miR-494- | | |
| 002365 | | 002365 | 002365 | | |
| | mmu-miR-451- | hsa-miR-451a | | | |
| | 001141 | | | | |
| hsa-miR-31- | | | | | |
| 002279 | | | | | |
| — | hsa-miR-436- | | hsa-miR-436- | | |
| | 001273 | | 001273 | | |
| hsa-miR-374- | | | | hsa-miR-374- | hsa-miR-374- |
| 000563 | | | | 000563 | 000563 |
| — | | | | hsa-miR-20b- | hsa-miR-20b- |
| | | | | 001014 | 001014 |
| hsa-miR-126- | hsa-miR-126- | | | hsa-miR-126- | hsa-miR-126- |
| 002228 | 002228 | | | 002228 | 002228 |
| hsa-miR-376c- | hsa-miR-376c- | | | hsa-miR-376c- | hsa-miR-376c- |
| 002122 | 002122 | | | 002122 | 002122 |
| hsa-miR-126#- | hsa-miR-126#- | | | hsa-miR-126#- | hsa-miR-126#- |
| 000451 | 000451 | | | 000451 | 000451 |
| hsa-miR-186- | | | | hsa-miR-186- | hsa-miR-186- |
| 002285 | | | | 002285 | 002285 |
| hsa-miR-146b- | | | | hsa-miR-146b- | hsa-miR-146b- |
| 001097 | | | | 001097 | 001097 |
| hsa-miR-30c- | | | | hsa-miR-30c- | hsa-miR-30c- |
| 000419 | | | | 000419 | 000419 |
| hsa-miR-875-5p- | | | hsa-miR-875-5p- | | |
| 002203 | | | 002203 | | |
| | | rno-miR-7#- | | rno-miR-7#- | |
| | | 001338 | | 001338 | |
| | | | | hsa-miR-199a-3p- | hsa-miR-199a-3p- |
| | | | | 002304 | 002304 |
| | | hsa-miR-19b | | hsa-miR-19b- | hsa-miR-19b- |
| | | | | 000396 | 000396 |
| | | | | hsa-miR-30b- | hsa-miR-30b- |
| | | | | 000602 | 000602 |
| | | hsa-miR-21 | | hsa-miR-21- | hsa-miR-21- |
| | | | | 000397 | 000397 |
| | | hsa-miR-20a | | hsa-miR-20a- | hsa-miR-20a- |
| | | | | 000580 | 000580 |
| | | hsa-miR-26b | | hsa-miR-26b- | hsa-miR-26b- |
| | | | | 000407 | 000407 |
| | hsa-miR-221- | | | | hsa-miR-221- |
| | 000524 | | | | 000524 |
| hsa-miR-432- | | | hsa-miR-432- | | |
| 001026 | | | 001026 | | |
| hsa-miR-150- | | | hsa-miR-150- | hsa-miR-150- | |
| 000473 | | | 000473 | 000473 | |
| | | | | hsa-let-7e- | hsa-let-7e- |
| | | | | 002406 | 002406 |
| | | | | hsa-miR-454- | hsa-miR-454- |
| | | | | 002323 | 002323 |
| | | | hsa-let-7a- | | |
| | | | 000377 | | |
| | | | — | hsa-miR-17- | hsa-miR-17- |
| | | | | 002308 | 002303 |
| | | | hsa-miR-130a- | | hsa-miR-130a- |
| | | | 000454 | | 000454 |
| | | mmu-miR-495- | | mmu-miR-495- | mmu-miR-495- |
| | | 001663 | | 001663 | 001663 |
| | | hsa-miR-191 | | | hsa-miR-191 |
| | | | hsa-miR-516-3p | | |
| | | | hsa-miR-522 | | |
| | | | hsa-miR-574-3p | | hsa-miR-574-3p |

lower-expression
over-expression

TABLE VI

| | R5 | | X4 | | Dual | | Control | |
|---|---|---|---|---|---|---|---|---|
| | mean Ct | sd | mean Ct | sd | mean Ct | sd | mean Ct | sd |
| hsa-let-7a | 30.348 | 2.563 | 30.591 | 1.016 | 29.859 | 1.127 | 30.733 | 1.211 |
| hsa-let-7e | 28.011 | 0.994 | 27.717 | 0.510 | 26.494 | 0.571 | 27.279 | 0.731 |
| hsa-miR-124-3p | 27.379 | 0.657 | 27.251 | 1.406 | 27.457 | 0.763 | 29.804 | 0.639 |
| (mmu-miR-124a) | | | | | | | | |
| hsa-miR-126 | 26.604 | 0.755 | 26.157 | 0.988 | 24.350 | 1.120 | 24.905 | 0.695 |
| hsa-miR-126# | 27.373 | 0.774 | 26.793 | 0.999 | 25.138 | 1.199 | 25.499 | 0.637 |
| hsa-miR-130a | 29.965 | 0.846 | 31.112 | 1.766 | 28.959 | 1.023 | 29.843 | 1.067 |
| hsa-miR-146b | 26.326 | 1.032 | 25.265 | 0.638 | 24.380 | 0.794 | 24.766 | 0.537 |
| hsa-miR-150 | 21.833 | 0.702 | 20.701 | 0.480 | 20.575 | 0.384 | 20.422 | 0.508 |
| hsa-miR-17 | 24.294 | 0.467 | 23.964 | 0.566 | 23.067 | 0.495 | 23.531 | 0.454 |
| hsa-miR-186 | 27.791 | 0.642 | 26.907 | 0.510 | 26.122 | 0.532 | 26.301 | 0.589 |
| hsa-miR-191 | 23.474 | 0.496 | 23.372 | 0.474 | 22.452 | 0.390 | 23.262 | 0.519 |
| hsa-miR-199a-3p | 30.459 | 1.324 | 30.434 | 1.384 | 28.503 | 0.864 | 29.203 | 0.785 |
| hsa-miR-19b | 25.806 | 0.644 | 25.747 | 0.744 | 24.395 | 0.636 | 25.208 | 0.446 |
| hsa-miR-20a | 26.883 | 0.465 | 26.929 | 0.474 | 25.655 | 0.580 | 26.501 | 0.455 |
| hsa-miR-20b | 29.031 | 0.950 | 28.180 | 0.537 | 27.169 | 0.659 | 27.892 | 1.042 |
| hsa-miR-21 | 28.602 | 0.592 | 28.997 | 0.871 | 27.355 | 0.414 | 28.381 | 0.616 |
| hsa-miR-221 | 29.180 | 0.841 | 30.092 | 1.249 | 28.012 | 0.819 | 28.781 | 1.042 |
| hsa-miR-26b | 26.043 | 0.916 | 25.336 | 0.592 | 24.174 | 0.812 | 25.143 | 0.786 |
| hsa-miR-30b | 27.597 | 0.670 | 27.391 | 0.597 | 26.269 | 0.523 | 26.924 | 0.435 |
| hsa-miR-30c | 27.200 | 0.642 | 26.631 | 0.625 | 25.641 | 0.654 | 26.202 | 0.504 |
| hsa-miR-31 | 29.812 | 1.592 | 28.347 | 1.027 | 28.144 | 1.560 | 27.368 | 0.832 |
| hsa-miR-374 | 28.699 | 0.801 | 28.129 | 0.660 | 26.773 | 0.743 | 27.496 | 0.583 |
| hsa-miR-376c | 33.851 | 2.427 | 32.515 | 1.402 | 30.392 | 1.392 | 30.622 | 1.106 |
| hsa-miR-432 | 32.914 | 2.781 | 31.605 | 2.031 | 31.577 | 2.393 | 30.233 | 0.905 |
| hsa-miR-451a | 30.234 | 1.869 | 29.016 | 0.859 | 28.673 | 1.448 | 30.550 | 0.846 |
| (mmu-miR-451) | | | | | | | | |
| hsa-miR-454 | 28.741 | 0.573 | 28.407 | 0.527 | 27.330 | 0.647 | 28.149 | 0.560 |
| hsa-miR-486 | 26.877 | 1.079 | 25.603 | 0.728 | 25.777 | 0.805 | 26.500 | 0.622 |
| hsa-miR-494 | 29.055 | 0.671 | 29.865 | 0.553 | 29.002 | 0.668 | 30.702 | 0.462 |
| hsa-miR-495 | 34.227 | 2.273 | 34.372 | 1.102 | 31.703 | 1.167 | 33.030 | 1.887 |
| (mmu-miR-495) | | | | | | | | |
| hsa-miR-516-3p | 29.356 | 0.792 | 30.259 | 1.074 | 29.706 | 1.593 | 30.019 | 0.844 |
| hsa-miR-522 | 31.588 | 1.801 | 33.855 | 1.760 | 33.560 | 2.349 | 33.049 | 1.262 |
| hsa-miR-574-3p | 28.429 | 0.388 | 28.954 | 0.671 | 28.117 | 0.313 | 28.448 | 0.399 |
| hsa-miR-7-1-3p | 32.585 | 1.842 | 31.528 | 1.232 | 29.986 | 0.731 | 31.658 | 0.912 |
| (rno-miR-7#) | | | | | | | | |
| hsa-miR-875-5p | 29.918 | 1.055 | 31.048 | 1.241 | 30.002 | 1.021 | 31.240 | 0.606 |

Ct values obtained by RTqPCR on array cards

1. An in vitro method for determining co-receptor usage of a virus in an infected subject, comprising (i) determining the presence or level of at least one circulating microRNA in a biological sample from the subject, and correlating said determination to the co-receptor usage of the virus, wherein said circulating microRNA is selected from hsa-let-7f-2#; hsa-miR-106a; hsa-miR-1208; hsa-miR-1233; hsa-miR-1243; hsa-miR-1262; hsa-miR-1267; hsa-miR-1276; hsa-miR-1285; hsa-miR-1290; hsa-miR-1298; hsa-miR-1305; hsa-miR-140-3p; hsa-miR-142-3p; hsa-miR-144#; hsa-miR-146a; hsa-miR-150; hsa-miR-17; hsa-miR-186; hsa-miR-20a; hsa-miR-210; hsa-miR-222; hsa-miR-223; hsa-miR-24; hsa-miR-24-2#; hsa-miR-26b; hsa-miR-30a-3p; hsa-miR-30a-5p; hsa-miR-30c; hsa-miR-320; hsa-miR-323-3p; hsa-miR-338-5P; hsa-miR-33a; hsa-miR-33b; hsa-miR-340; hsa-miR-342-3p; hsa-miR-378; hsa-miR-424; hsa-miR-483-5p; hsa-miR-484; hsa-miR-485-3p; hsa-miR-486; hsa-miR-486-3p; hsa-miR-502; hsa-miR-550; hsa-miR-557; hsa-miR-572; hsa-miR-575; hsa-miR-581; hsa-miR-582-3p; hsa-miR-584; hsa-miR-586; hsa-miR-596; hsa-miR-597; hsa-miR-625#; hsa-miR-630; hsa-miR-638; hsa-miR-645; hsa-miR-648; hsa-miR-656; hsa-miR-657; hsa-miR-659; hsa-miR-661; hsa-miR-720; hsa-miR-770-5p; hsa-miR-875-5p; hsa-miR-892b; hsa-miR-99b#; mmu-miR-451; or mmu-miR-93; and (ii) adjusting the treatment of said subject based on said co-receptor usage.

2. (canceled)

3. The method of claim 1, comprising determining in the sample the circulating level of at least one microRNA selected hsa-miR-106a; hsa-miR-1208; hsa-miR-1233; hsa-miR-1267; hsa-miR-1290; hsa-miR-146a; hsa-miR-150; hsa-miR-17; hsa-miR-186; hsa-miR-20a; hsa-miR-222; hsa-miR-223; hsa-miR-24; hsa-miR-26b; hsa-miR-30a-5p; hsa-miR-30c; hsa-miR-323-3p; hsa-miR-338-5P; hsa-miR-33b; hsa-miR-342-3p; hsa-miR-378; hsa-miR-424; hsa-miR-483-5p; hsa-miR-484; hsa-miR-486; hsa-miR-486-3p; hsa-miR-572; hsa-miR-596; hsa-miR-625#; hsa-miR-638; hsa-miR-659; hsa-miR-661; hsa-miR-99b#; or mmu-miR-93.

4. (canceled)

5. The method of claim 1, wherein the biological sample is a biological fluid.

6. The method of claim 5, wherein the biological sample is selected from blood, plasma, serum, saliva or urine.

7. The method of claim 1, wherein step (i) comprises a determination of the presence or level of at least one microRNA in circulating PBMCs from the subject, and correlating said determination to the co-receptor usage of the virus, wherein the at least one microRNA is selected from hsa-let-7a; hsa-let-7d; hsa-let-7e; hsa-let-7f; hsa-let-7g; hsa-miR-103; hsa-miR-106a; hsa-miR-106b; hsa-miR-124-3p (mmu-miR-124a); hsa-miR-1244; hsa-miR-126; hsa-miR-126#; hsa-miR-1260; hsa-miR-1274A; hsa-miR-1276; hsa-miR-130a; hsa-miR-130b; hsa-miR-133a; hsa-miR-134 (mmu-miR-134); hsa-miR-139-5p; hsa-miR-140-5p (mmu-miR-140); hsa-miR-142-3p; hsa-miR-142-5p; hsa-miR-146a; hsa-miR-146b; hsa-miR-148a; hsa-miR-148b; hsa-miR-149#; hsa-miR-150; hsa-miR-151-5P; hsa-miR-15b; hsa-miR-16; hsa-miR-17; hsa-miR-1825; hsa-miR-185; hsa-miR-186; hsa-miR-18b; hsa-miR-191; hsa-miR-192; hsa-miR-195; hsa-miR-196b; hsa-miR-199a-3p; hsa-miR-19a; hsa-miR-19b; hsa-miR-200b; hsa-miR-20a; hsa-miR-20b; hsa-miR-21; hsa-miR-221; hsa-miR-223; hsa-miR-223#; hsa-miR-24-2#; hsa-miR-25; hsa-miR-26a; hsa-miR-26b; hsa-miR-27a; hsa-miR-27a#; hsa-miR-28; hsa-miR-299-3p; hsa-miR-29a; hsa-miR-29c; hsa-miR-301; hsa-miR-301b; hsa-miR-30b; hsa-miR-30c; hsa-miR-31; hsa-miR-324-3p; hsa-miR-328; hsa-miR-335; hsa-miR-340; hsa-miR-340#; hsa-miR-342-3p; hsa-miR-365; hsa-miR-374; hsa-miR-374b-5p (mmu-miR-374-5p); hsa-miR-376c; hsa-miR-380-5p; hsa-miR-410; hsa-miR-422a; hsa-miR-425-5p; hsa-miR-432; hsa-miR-451a (mmu-miR-451); hsa-miR-454; hsa-miR-486; hsa-miR-494; hsa-miR-495 (mmu-miR-495); hsa-miR-516-3p; hsa-miR-522; hsa-miR-532; hsa-miR-532-3p; hsa-miR-543; hsa-miR-571; hsa-miR-574-3p; hsa-miR-590-3P; hsa-miR-590-5p; hsa-miR-628-5p; hsa-miR-638; hsa-miR-642; hsa-miR-650; hsa-miR-651; hsa-miR-652; hsa-miR-660; hsa-miR-7-1-3p (rno-miR-7#); hsa-miR-744#; hsa-miR-765; hsa-miR-875-5p; hsa-miR-93-5p (mmu-miR-93); or hsa-miR-942.

8. (canceled)

9. The method of claim 7, comprising determining in circulating PBMCs the level of at least one microRNA selected from hsa-let-7a; hsa-let-7e; hsa-miR-124-3p (mmu-miR-124a); hsa-miR-126; hsa-miR-126#; hsa-miR-130a; hsa-miR-146b; hsa-miR-150; hsa-miR-17; hsa-miR-186; hsa-miR-191; hsa-miR-199a-3p; hsa-miR-19b; hsa-miR-20a; hsa-miR-20b; hsa-miR-21; hsa-miR-221; hsa-miR-26b; hsa-miR-30b; hsa-miR-30c; hsa-miR-31; hsa-miR-374; hsa-miR-376c; hsa-miR-432; hsa-miR-451a (mmu-miR-451); hsa-miR-454; hsa-miR-486; hsa-miR-494; hsa-miR-495 (mmu-miR-495); hsa-miR-516-3p; hsa-miR-522; hsa-miR-574-3p; hsa-miR-7-1-3p (rno-miR-7#); or hsa-miR-875-5p.

10. The method of claim 1, comprising determining the level of at least 2, 3, 4, 5, 6, 7, 8, 9, or 10 of said microRNAs to obtain a profile, and correlating said profile to the co-receptor usage of the virus.

11. The method of claim 1, wherein the virus is human immunodeficiency virus (HIV).

12. The method of claim 11, for determining whether the HIV in said subject uses the CXCR4 or the CCR5 co-receptor.

13. The method of claim 12, wherein step (i) comprises determining the level of circulating miR-638 in a sample from the infected subject and comparing said level to a reference level, an increase in said level being indicative of CXCR4 co-receptor usage by the virus.

14. The method of claim 1, comprising determining, in a sample from the infected subject, the level of one of the following combinations of circulating microRNAs: hsa-miR-661 and hsa-miR-638; hsa-miR-30a-5p and hsa-miR-638; hsa-miR-661 and hsa-miR-30a-5p; hsa-miR-661, hsa-miR-638 and hsa-miR-30a-5p; hsa-miR-661, hsa-miR-638 and hsa-miR-659; or hsa-miR-661, hsa-miR-638, hsa-miR-30a-5p, hsa-miR-659, hsa-miR-596, hsa-miR-1233, hsa-miR-572 and hsa-miR-625#.

15. The method of claim 1, comprising determining, in a PBMC sample from the infected subject, the level of one of the following combinations of microRNAs: hsa-miR-150 and hsa-miR-486; hsa-miR-150, hsa-miR-486, hsa-miR-522 and hsa-miR-574-3p; hsa-miR-150, hsa-miR-486 and hsa-miR-522; hsa-miR-150, hsa-miR-486 and hsa-miR-574-3p; hsa-miR-126 and hsa-miR-146b; hsa-miR-126, hsa-miR-146b, and hsa-miR-20a; hsa-miR-126 and hsa-miR-20a; hsa-miR-146b and (hsa-miR-20a or hsa-miR-21 or hsa-miR-376c or hsa-let-7e); or hsa-miR-126, hsa-miR-146b, hsa-miR-20a, hsa-miR-21, hsa-miR-376c and hsa-let-7e.

16. The method of claim 1, wherein the microRNA is detected or determined in the sample by hybridization, amplification, ligand-binding, or a functional assay.

17. The method of claim 16, which comprises contacting an aliquot of the biological sample with a nucleic acid probe, a nucleic acid primer, or a ligand, characteristic of said at least one microRNA, and determining the presence or amount of complexes formed between said probe, primer, or ligand and nucleic acids in the aliquot.

18. The method of claim 17, wherein the probe or ligand is immobilized on a support.

19. The method of claim 1, wherein the biological sample is treated prior to the determination, preferably by dilution, or concentration, or enrichment for microRNAs, or for removing cells or protecting RNA.

20. The method of claim 1, wherein the biological sample has been frozen and is thawed prior to the determination.

21. The method of claim 1, wherein the determination is performed less than 48 hours after sample collection, treatment or thawing, preferably less than 24 hours.

22. The method of claim 1, wherein the subject is a human subject.

23. A microarray comprising a plurality of nucleic acid probes, said plurality of probes comprising distinct probes specific for at least 2, preferably at least 3, more preferably at least 5 distinct microRNAs selected from hsa-let-7f-2#; hsa-miR-106a; hsa-miR-1208; hsa-miR-1233; hsa-miR-1243; hsa-miR-1262; hsa-miR-1267; hsa-miR-1276; hsa-miR-1285; hsa-miR-1290; hsa-miR-1298; hsa-miR-1305; hsa-miR-140-3p; hsa-miR-142-3p; hsa-miR-144#; hsa-miR-146a; hsa-miR-150; hsa-miR-17; hsa-miR-186; hsa-miR-20a; hsa-miR-210; hsa-miR-222; hsa-miR-223; hsa-miR-24; hsa-miR-24-2#; hsa-miR-26b; hsa-miR-30a-3p; hsa-miR-30a-5p; hsa-miR-30c; hsa-miR-320; hsa-miR-323-3p; hsa-miR-338-5P; hsa-miR-33a; hsa-miR-33b; hsa-miR-340; hsa-miR-342-3p; hsa-miR-378; hsa-miR-424; hsa-miR-483-5p; hsa-miR-484; hsa-miR-485-3p; hsa-miR-486; hsa-miR-486-3p; hsa-miR-502; hsa-miR-550; hsa-miR-557; hsa-miR-572; hsa-miR-575; hsa-miR-581; hsa-miR-582-3p; hsa-miR-584; hsa-miR-586; hsa-miR-596; hsa-miR-597; hsa-miR-625#; hsa-miR-630; hsa-miR-638; hsa-miR-645; hsa-miR-648; hsa-miR-656; hsa-miR-657; hsa-miR-659; hsa-miR-661; hsa-miR-720; hsa-miR-770-5p; hsa-miR-875-5p; hsa-miR-892b; hsa-miR-99b#; mmu-miR-451; or mmu-miR-93.

24. (canceled)

25. A microarray comprising a plurality of nucleic acid probes, said plurality of probes comprising distinct probes specific for at least 2, preferably at least 3, more preferably at least 5 distinct microRNAs selected from hsa-let-7a; hsa-let-7d; hsa-let-7e; hsa-let-7f; hsa-let-7g; hsa-miR-103; hsa-miR-106a; hsa-miR-106b; hsa-miR-124-3p (mmu-miR-124a); hsa-miR-1244; hsa-miR-126; hsa-miR-126#; hsa-miR-1260; hsa-miR-1274A; hsa-miR-1276; hsa-miR-130a; hsa-miR-130b; hsa-miR-133a; hsa-miR-134 (mmu-miR-134); hsa-miR-139-5p; hsa-miR-140-5p (mmu-miR-140); hsa-miR-142-3p; hsa-miR-142-5p; hsa-miR-146a; hsa-miR-146b; hsa-miR-148a; hsa-miR-148b; hsa-miR-149#; hsa-miR-150; hsa-miR-151-5P; hsa-miR-15b; hsa-miR-16; hsa-miR-17; hsa-miR-1825; hsa-miR-185; hsa-miR-186; hsa-miR-18b; hsa-miR-191; hsa-miR-192; hsa-miR-195; hsa-miR-196b; hsa-miR-199a-3p; hsa-miR-19a; hsa-miR-19b; hsa-miR-200b; hsa-miR-20a; hsa-miR-20b; hsa-miR-21; hsa-miR-221; hsa-miR-223; hsa-miR-223#; hsa-miR-24-2#; hsa-miR-25; hsa-miR-26a; hsa-miR-26b; hsa-miR-27a; hsa-miR-27a#; hsa-miR-28; hsa-miR-299-3p; hsa-miR-29a; hsa-miR-29c; hsa-miR-301; hsa-miR-301b; hsa-miR-30b; hsa-miR-30c; hsa-miR-31; hsa-miR-324-3p; hsa-miR-328; hsa-miR-335; hsa-miR-340; hsa-miR-340#; hsa-miR-342-3p; hsa-miR-365; hsa-miR-374; hsa-miR-374b-5p (mmu-miR-374-5p); hsa-miR-376c; hsa-miR-380-5p; hsa-miR-410; hsa-miR-422a; hsa-miR-425-5p; hsa-miR-432; hsa-miR-451a (mmu-miR-451); hsa-miR-454; hsa-miR-486; hsa-miR-494; hsa-miR-495 (mmu-miR-495); hsa-miR-516-3p; hsa-miR-522; hsa-miR-532; hsa-miR-532-3p; hsa-miR-543; hsa-miR-571; hsa-miR-574-3p; hsa-miR-590-3P; hsa-miR-590-5p; hsa-miR-628-5p; hsa-miR-638; hsa-miR-642; hsa-miR-650; hsa-miR-651; hsa-miR-652; hsa-miR-660; hsa-miR-7-1-3p (rno-miR-7#); hsa-miR-744#; hsa-miR-765; hsa-miR-875-5p; hsa-miR-93-5p (mmu-miR-93); or hsa-miR-942.

26. (canceled)

27. A set of nucleic acid primers comprising a plurality of nucleic acid primers, said plurality comprising primers that specifically amplify at least 2, preferably at least 3, more preferably at least 5 distinct microRNAs selected from hsa-let-7f-2#; hsa-miR-106a; hsa-miR-1208; hsa-miR-1233; hsa-miR-1243; hsa-miR-1262; hsa-miR-1267; hsa-miR-1276; hsa-miR-1285; hsa-miR-1290; hsa-miR-1298; hsa-miR-1305; hsa-miR-140-3p; hsa-miR-142-3p; hsa-miR-144#; hsa-miR-146a; hsa-miR-150; hsa-miR-17; hsa-miR-186; hsa-miR-20a; hsa-miR-210; hsa-miR-222; hsa-miR-223; hsa-miR-24; hsa-miR-24-2#; hsa-miR-26b; hsa-miR-30a-3p; hsa-miR-30a-5p; hsa-miR-30c; hsa-miR-320; hsa-miR-323-3p; hsa-miR-338-5P; hsa-miR-33a; hsa-miR-33b; hsa-miR-340; hsa-miR-342-3p; hsa-miR-378; hsa-miR-424; hsa-miR-483-5p; hsa-miR-484; hsa-miR-485-3p; hsa-miR-486; hsa-miR-486-3p; hsa-miR-502; hsa-miR-550; hsa-miR-557; hsa-miR-572; hsa-miR-575; hsa-miR-581; hsa-miR-582-3p; hsa-miR-584; hsa-miR-586; hsa-miR-596; hsa-miR-597; hsa-miR-625#; hsa-miR-630; hsa-miR-638; hsa-miR-645; hsa-miR-648; hsa-miR-656; hsa-miR-657; hsa-miR-659; hsa-miR-661; hsa-miR-720; hsa-miR-770-5p; hsa-miR-875-5p; hsa-miR-892b; hsa-miR-99b#; mmu-miR-451; or mmu-miR-93.
